(19) United States
(12) Patent Application Publication
(10) Pub. No.: US 2024/0350489 A1
(43) Pub. Date: Oct. 24, 2024

(54) LOU064 FOR TREATING MULTIPLE SCLEROSIS

(71) Applicant: NOVARTIS AG, Basel (CH)

(72) Inventors: Souvik BHATTACHARYA, Weymouth, MA (US); Bruno BIETH, Saint-Louis (FR); Bruno CENNI, Sissach (CH); Peter END, Oberwil (CH); Gordon GRAHAM, Huningue (FR); Michael JUHNKE, Basel (CH); Rajesh Singh KARAN, Reinach (CH); Allison Donna MANN, Fort Lee, NJ (US); Etienne PIGEOLET, Rantzwiller (FR); Karin RAPP, Lörrach (DE); Kim-Hien SIN, Saint-Louis (FR); Huixin YU, Bottmingen (CH); Ying ZHANG, Morris Plains, NJ (US)

(21) Appl. No.: 18/685,551

(22) PCT Filed: Sep. 1, 2022
(86) PCT No.: PCT/IB2022/058205

§ 371 Date: Feb. 22, 2024

Related U.S. Application Data
(60) Provisional application No. 63/240,444, filed on Sep. 3, 2021.
(60) Provisional application No. 63/282,363, filed on Nov. 23, 2021.
(60) Provisional application No. 63/304,106, filed on Jan. 28, 2022.
(60) Provisional application No. 63/353,866, filed on Jun. 21, 2022.
(60) Provisional application No. 63/369,008, filed on Jul. 21, 2022.

(51) Int. Cl. A61K 31/505; A61K 9/20; A61K 9/48; A61P 25/28

(52) U.S. Cl. CPC A61K 31/505; A61K 9/2018; A61K 9/2027; A61K 9/2054; A61K 9/4858; A61K 9/4866; A61P 25/28

(57) ABSTRACT

The invention concerns method of treating multiple sclerosis (MS) by administering a therapeutically effective dose of LOU064 or a pharmaceutically acceptable salt thereof.

### FIELD OF THE INVENTION

The invention concerns LOU064 or a pharmaceutically acceptable salt thereof for use in the effective treatment of multiple sclerosis (MS).

### BACKGROUND OF THE INVENTION

Multiple Sclerosis (MS) is a chronic, immune-mediated disease of the central nervous system characterized by inflammation, demyelination and axonal/neuronal destruction, ultimately leading to severe disability. Although there is no cure for the disease, a variety of disease-modifying therapies (DMT) are available which usually slow down disease progression.

Although most disease-modifying therapies for MS have traditionally been conceptualized as functioning via T cell-based mechanisms, a growing body of data indicates that these DMTs have demonstrable effects on B cells as well. Common themes include promoting naive B cells rather than memory or plasmablasts (alemtuzumab); shifting B-cell cytokines towards an anti-inflammatory tone (beta interferon, glatiramer acetate, fingolimod); increasing B-regs (beta interferon, glatiramer acetate, fingolimod and dimethyl fumarate); decreasing class II MHC expression and costimulatory molecules on B cells required for antigen presentation (beta interferon and dimethyl fumarate); sequestering B cells in lymphoid organs (fingolimod); blocking VLA-4 mediated B cell trafficking to the CNS (natalizumab); or direct cytolysis of B cells (alemtuzumab, teriflunomide, mitoxantrone), see Greenfield et al., Ann. Neurol. 2018 January; 83(1): 13-26.

While these DMTs usually significantly reduce relapse rates and MRI disease activity and thus delay the time to disability worsening, generally (severe) adverse events may be associated with each of these DMTs. For example, natalizumab may lead to an increased risk of a fatal opportunistic infection (i.e. progressive multifocal leukoencephalopathy or PML), while some oral DMTs may be associated with SiP-related safety risks, e.g. bradyarrhythmias upon treatment initiation, macular edema, hypertension and liver transaminase elevations.

Another treatment option for patients with MS is the unspecific depletion of B cells by administration of monoclonal antibodies that target CD20-expressing B cells such as ofatumumab, ocrelizumab, rituximab, obinutuzumab and ublituximab, see Torke, S. and Weber, M. S. (2020), Expert Opinion on Investigational Drugs, 29:10, 1143-1150.

However, long-term trials have highlighted that with an increasing life-time dose of B-cell-depleting agents, key functions of the immune system can be impaired.

Nevertheless, in light of the long-term effects of lasting immunosuppressive therapies, such as the decline in humoral competence, sustainable and flexible approaches to control MS-driving pathogenic B cells are needed, particularly giving the doctors flexibility in the treatment regimen.

Recently, the therapeutic inhibition of Bruton's tyrosine kinase (BTK) has been suggested as a novel strategy towards achieving this goal.

BTK is an enzyme centrally involved in B cell-receptor (BCR) signaling and is essential for normal B cell maturation. Although BTK's main role is described to mediate BCR signaling, it has since been shown to be involved in other pathways such as Fc-receptor (FcR) and toll-like receptor (TLR) signaling as well as in the production of reactive oxygen species (ROS). BTK belongs to the TEC (tyrosine kinase expressed in hepatocellular carcinoma) family of kinases. The expression of the members of the TEC family of kinases is mainly restricted to the hematopoietic system.

BTK is essential for normal B cell development and maturation. The absence of BTK, in for example X-linked agammaglobulinemia (XLA) patients, reveals an almost complete lack of peripheral B and plasma cells resulting in very low levels of circulating immunoglobulins. In contrast, in xid mice, there is a specific arrest of peripheral B cell maturation, while the numbers of pre-B cells generated in the bone marrow (BM) are normal. BTK is crucial for the progression of pre-B cells by controlling the IL-7 driven expansion of large cycling pre-B cells as well as by promoting their progression to small resting pre-B cells. Later on, BTK controls the expression of the first immunoglobulin chains as well as the entry of B cells into follicular structures. Finally, BTK is involved in BCR-mediated B cell activation and their ultimate, terminal differentiation into memory or plasma cells.

Thus, without wishing to be bound by any theory, BTK inhibitors which block a critical enzyme involved in B cell maturation will inhibit pathogenic B cells in diseases like MS.

Up to date, several BTK inhibitors have been developed and tested for the treatment of a number of diseases. Thus, ibrutinib (Imbruvica), is approved for the treatment of chronic lymphocytic leukemia (CLL), Waldenstrom's macroglobulinemia and is a second-line treatment for mantle cell lymphoma (MCL), marginal zone lymphoma, and chronic graft-vs-host disease. Also approved for the therapy of MCL are acalabrutinib (Calquence) and zanubrutinib (Brukinsa). Acalabrutinib and Zanubrutinib as well as the novel compounds ONO-4059 (Tirabrutinib), HM71224 (Poseltinib) and ABBV-105 (Upadacitinib) are currently being tested for their efficacy in B cell malignancies and/or autoimmune diseases such as rheumatoid arthritis (RA), Sjögren's Syndrome (SjS) and systemic lupus erythematosus (SLE).

Up to date, the BTK inhibitors evobrutinib, tolebrutinib and fenebrutinib have entered phase III studies in MS patients, orelabrutinib is tested in a phase II study and BIIB091 was tested in a phase I study for efficacy in the treatment of MS.

Evobrutinib and tolebrutinib are classified as covalent, irreversible BTK inhibitors, while the BTKi binding mechanism of fenebrutinib is described as non-covalent, reversible.

Tolebrutinib brought about a reduction in MS lesion development in its mid-stage trials, with headaches and cold-like symptoms being the most frequent adverse events (Dolgin, E. BTK blockers make headway in multiple sclerosis. Nat. Biotechnol. 39, 3-5 (2021)).

Evobrutinib has been tested in animal models as well as clinical trials for RA, SLE and a phase II safety and efficacy study in RRMS.

In experimental autoimmune encephalomyelitis (EAE), an animal model of MS, evobrutinib was effective at a dose of 3 mg/kg, while a dose of 10 mg/kg did not further improve the effect, supporting the notion of complete BTK inhibition at 3 mg/kg (Torke, S. et al., Inhibition of Bruton's tyrosine kinase interferes with pathogenic B-cell development in inflammatory CNS demyelinating disease, Acta Neuropathol. 140, 535-548 (2020)).

In a phase II trial of evobrutinib as a monotherapy for RRMS (relapsing-remitting multiple sclerosis) and active secondary progressive MS 3 doses of evobrutinib (25 mg once daily, 75 mg once daily or 75 mg twice daily) were tested against placebo or dimethyl fumarate (DMF). Patients with relapsing MS who received higher doses of evobrutinib of 75 mg once or twice per day showed a trend toward developing fewer brain lesions and experiencing fewer relapses compared with those given a placebo or dimethyl fumarate. However, apart from nasopharyngitis, higher evobrutinib doses of 75 mg once daily and twice daily were associated with a higher frequency of elevations of alanine aminotransferase (ALT), aspartate aminotransferase (AST) and lipase, thus raising safety issues (Montalban X. et al., 2019, N Engl J Med; 380(25): 2406-17).

Currently, a phase III trial evaluating the efficacy and safety of evobrutinib administered orally twice daily versus teriflunomide, administered orally once daily in participants with relapsing multiple sclerosis (RMS) is in progress.

Considering the potential adverse events of these BTK inhibitors and not fully established effectivity, there is the need for improved BTK inhibitors that will be effective and safe, particularly in the long-term treatment of MS.

### BRIEF DESCRIPTION OF FIGURES

FIG. 1

Effect of prophylactic LOU064 (3 mg/kg p.o. b.i.d. and 30 mg/kg p.o. b.i.d.) on daily neurological deficits during HumanMOG EAE. Daily body weight change (A) was significantly improved with LOU064 30 mg/kg p.o. b.i.d. treatment, suggesting a reduction in inflammation-induced cachexia. Consistent with the body weight data was an improvement in EAE scores with LOU064 30 mg/kg p.o. b.i.d. (B). Group sizes n=8-10 per treatment. Statistical significance determined using Kruskal-Wallis with Dunn's test (non-parametric ANOVA).

FIG. 2

Prophylactic LOU064 30 mg/kg (b.i.d.) significantly reduced disease incidence during HumanMOG EAE. LOU064 b.i.d. treatment reduced the frequency and speed of EAE onset. Group sizes n=10 per treatment. Statistical significance determined using Kaplan-Meier non-parametric test.

FIG. 3

Prophylactic LOU064 dose-dependently reduced disease burden during HumanMOG EAE. LOU064 30 mg/kg b.i.d. treatment significantly reduced the peak EAE score (A) and cumulative disease burden (B). Group sizes n=10 per treatment. Statistical significance determined using Kruskal-Wallis with Dunn's test (non-parametric ANOVA).

FIG. 4

BTK occupancy in spleen at 1, 5 and 8 hours after the last dose of oral b.i.d. dosing of LOU064. Shown are the occupancy levels of individual animals. The group means are shown as the central bars and the standard deviations are shown as whisker error bars. Statistical significance (ANOVA, followed by Dunnett's test) is shown as **** for p<0.0001. One outlier in the vehicle group was removed after Grubbs' test (with −112% BTK occupancy).

FIG. 5

BTK occupancy in lymph nodes at 1, 5 and 8 hours after the last dose of oral b.i.d. dosing of LOU064. Shown are the occupancy levels of individual animals. The group means are shown as the central bars and the standard deviations are shown as whisker error bars. Statistical significance (ANOVA, followed by Dunnett's test) is shown as **** for p<0.0001.

FIG. 6

BTK occupancy in brain homogenate at 1, 5 and 8 hours after the last dose of oral b.i.d. dosing of LOU064. Shown are the occupancy levels of individual animals. The group means are shown as the central bars and the standard deviations are shown as whisker error bars. Statistical significance (ANOVA, followed by Dunnett's test) is shown as ** for p<0.01.

FIG. 7

Prophylactic LOU064 (b.i.d.) treatment during HumanMOG EAE only weakly modulates MOG-specific immunoglobulin responses. Oral LOU064 b.i.d. treatment significantly reduced MOG-specific IgM and IgG responses in serum; however, the effect was very weak (A, B). Subclass analysis revealed IgGI (C), IgG2a (D) and IgG2b (E) were reduced. No change was detected on IgG2c (F). Group sizes n=8-10 per treatment. Statistical significance (ANOVA with Dunnett's test) is shown as *p <0.05, **p<0.01, ***p<0.001, ****p<0.0001.

FIG. 8

Prophylactic LOU064 (b.i.d.) treatment during HumanMOG EAE improves inflammation-induced cachexia. There were no neurological symptoms observed in the pre-EAE phase of the model. (A). Oral LOU064 b.i.d. treatment (30 mg/kg twice daily) improved weight gain in the immunized mice suggesting that inflammation-induced cachexia was reduced (B). Group sizes n=5 per treatment.

FIG. 9

Prophylactic LOU064 (b.i.d.) treatment during HumanMOG EAE inhibits (auto)antigen recall proliferation. Isolated splenocytes and draining lymph node cells were incubated in vitro with HumanMOG for 48 hours. Antigen-specific proliferation was determined by [³H]-thymidine incorporation. Animals dosed in vivo with LOU064 demonstrated a dose-dependent reduction in MOG-specific proliferation (A, B) Polyclonal stimulation with anti-CD3/CD28 was unchanged (C). Group sizes n=5 per treatment.

FIG. 10

Prophylactic LOU064 (b.i.d.) treatment during HumanMOG EAE reduces Th17 cells. Mice immunized with HumanMOG and dosed in vivo b.i.d. with LOU064 demonstrated no significant changes in the frequencies of B-cell populations (A, B). The total CD4⁺ T-cell population was unchanged (C); however, intracellular cytokine staining revealed a significant reduction in Th17 (IL-17*) cells (D). No changes were observed in Th1 or Tregs (E,F). Group sizes n=5 per treatment. Statistical significance (ANOVA with Dunnett's test) is shown as *p<0.05.

FIG. 11

Prophylactic BTK inhibitors administered b.i.d. during RatMOG EAE demonstrates no adverse responses. Daily b.i.d. dosing of BTK inhibitors (LOU064, ibrutinib did not induced neurological symptoms (A) or worsen weight changes (B). Cyclosporin A (CsA) was associated with pronounced weight loss. Group sizes n=4-5 per treatment.

FIG. 12

Prophylactic BTK inhibitors administered b.i.d. during RatMOG EAE failed to inhibit (auto)antigen recall proliferation. Isolated draining lymph node cells were incubated in vitro with RatMOG for 48 hours. Antigen-specific proliferation was determined by [³H]-thymidine incorporation. Animals dosed in vivo with BTK inhibitors (LOU064, ibrutinib or Cyclosporin A (CsA) for 8 days. Group sizes n=4-5 per treatment.

FIG. 13

LOU064 reduced RatMOG EAE pathology. Daily body weight changes (A) were significantly reduced by LOU064 30 mg/kg p.o. b.i.d. treatment suggesting a reduction in inflammation-induced cachexia. Consistent with the body weight data was an improvement in EAE scores (B). Group sizes n=10 per treatment. Statistical significance determined using Kruskal-Wallis with Dunn's test (non-parametric ANOVA).

FIG. 14

LOU064 significantly reduced disease incidence during RatMOG EAE. LOU064 30 mg/kg b.i.d. treatment reduced the frequency and time of EAE onset (clinical score ≥1). Group sizes n=10 per treatment. Statistical significance determined using Kaplan-Meier non-parametric test.

FIG. 15

LOU064 reduced disease burden during RatMOG EAE. LOU064 30 mg/kg b.i.d. treatment significantly reduced the peak EAE score (A) and cumulative disease burden (B). Group sizes n=10 per treatment. Statistical significance determined using Kruskal-Wallis with Dunn's test (non-parametric ANOVA).

FIG. 16

Trough BTK occupancy in spleen, blood and brain. Trough BTK occupancy in spleen and blood indicate maximal target occupancy in the peak. BTK occupancy assessed in brain homogenates showed intermediate levels in spleen (A), blood (B) and brain (C), suggesting that significant but submaximal brain BTK occupancy was reached at peak. Statistical significance determined by two-sided unpaired t-test with Welch's correction.

FIG. 17

LOU064 did not modulate MOG-specific immunoglobulin responses during ratMOG EAE. Oral LOU064 b.i.d. treatment did not affect MOG-specific IgM (A) and IgG (B) responses in serum compared to vehicle. Subclass analysis revealed IgGI (C), IgG2a (D) and IgG2b (E), IgG2c (F) and IgG3 (G) were not modulated. Group sizes n=4-5 per treatment; p values determined between naive and vehicle-treated mice. Statistical significance analyzed with ANOVA (followed by Dunnett's test).

FIG. 18

NF-L concentration in serum is slightly decreased by LOU064 treatment (FIG. 18A) and is correlated with EAE clinical scores (FIG. 18B). Oral LOU064 b.i.d. treatment slightly reduced the mean serum NF-L level as compared to vehicle treated group. The NF-L levels in serum correlated with clinical scores observed at termination. UDL=upper detection limit; LDL=lower detection limit. Statistical difference determined with a simple linear regression test.

FIG. 19

BTK is expressed in lymph nodes and not in brain of naive mice. Histologic examination of lymph nodes and brain of naive mice is showing BTK expression in the lymph nodes (A), disseminated in the paracortex and in particular in B cell follicles (B). No BTK expression was detected in brain (C) and in the corpus callosum (D). BTK IHC staining with hematoxylin and bluing counterstaining.

FIG. 20

Preferred particle size distribution of nanosized LOU064.

FIG. 21

(A) Trough over 24 hours of BTK Occupancy at steady state. Graph showing the median prediction as point and the vertical lines showing the 95% prediction interval. (B) Average over 24 hours of BTK Occupancy at steady state. Graph showing the median prediction as point and the vertical lines showing the 95% prediction interval.

FIG. 22

Simulation of Spleen BTK occupancy at steady state.

FIG. 23

scRNA-sequence analysis of RatMOG EAE mice treated with LOU064: neuroinflammation signature is significantly downregulated in microglia from brains and spinal cords of EAE mice treated with LOU06.

### SUMMARY OF THE INVENTION

The problem underlying the present invention is to provide improved treatment strategies for MS patients, especially for long-term treatments. In particular, it is an object of the present invention to provide an advantageous MS therapy, preferably a highly effective MS therapy.

Another object is to provide an MS therapy that is as effective as a B cell-depleting therapy, in particular as effective as a CD19- and/or CD20-depleting therapy.

It is another object to provide an MS therapy that is as effective as a B cell-depleting therapy without undesirably influencing serum levels of immunoglobulins.

It is a further object to provide an MS therapy that is effective at reducing the annual relapse rate, in particular as effective at reducing the annual relapse rate as a B cell-depleting therapy.

A still further object is to provide an MS therapy that can delay the worsening of disability.

Another object is to provide an improved MS therapy, particularly exhibiting an improved safety and tolerability profile as compared to other approved oral disease modifying therapies and to B cell-depleting therapy, in particular as compared to a CD19-/CD20-depleting therapy.

The problem has unexpectedly been solved by the administration of LOU064 or a pharmaceutically acceptable salt thereof.

LOU064 (=N-(3-(6-amino-5-(2-(N-methylacrylamido)ethoxy)pyrimidin-4-yl)-5-fluoro-2-methylphenyl)-4-cyclopropyl-2-fluorobenzamide, INN: remibrutinib) is disclosed in WO 2015/079417 A1 as a drug candidate for the selective inhibition of Bruton's tyrosine kinase. This compound is a potent, highly selective, irreversible covalent BTK inhibitor. Due to binding to an inactive conformation of BTK, LOU064 exhibits an exquisite kinase selectivity and, thus, reduces kinase off-target binding and due to covalent inhibition, the compound exhibits a potent and sustained pharmacodynamic effect without the need for extended and high systemic compound exposure (Angst, D. et al., Discovery of LOU064 (Remibrutinib), a Potent and Highly Selective Covalent Inhibitor of Bruton's Tyrosine Kinase, J Med Chem. 2020 May 28; 63(10):5102-5118).

LOU064, which has previously been suggested for use in the treatment of chronic spontaneous urticaria (CSU) (WO2020/234782 A1) and Sjoegren's Syndrome (SjS) (WO2020/234781 A1), is currently being tested in phase II clinical studies for CSU and SjS.

In WO2020/234782 A1 b.i.d. administration of doses of 10 mg, 25 mg and 100 mg were generally suggested to reach maximal efficacy in CSU.

In a Phase 2b, randomized, double-blind, placebo-controlled trial evaluating the efficacy and safety of LOU064 over 12 weeks in patients inadequately controlled by Hi-antihistamines, with at least moderately active CSU, patients received LOU064 10 mg q.d. (once daily), 35 mg q.d., 100 mg q.d., 10 mg b.i.d. (twice daily), 25 mg b.i.d., 100 mg b.i.d., or placebo (1:1:1:1:1:1:1 ratio). The 25 mg b.i.d. regimen was found to be particularly effective compared to the other doses.

Hence, a dose of 25 mg b.i.d. was selected for a subsequent phase III clinical study for CSU.

BTK occupancy in blood and/or tissues has been reported to be a suitable biomarker for selecting doses for clinical studies such as CSU and SjS studies. (WO2020/234782 and WO2020/234781)

Furthermore, it has been reported that BTK occupancy and duration of BTK occupancy is different in blood and in various tissues in female rat. (WO2020/234781)

In preclinical studies assessing BTK occupancy of LOU064 already a single oral dose of 3 mg/kg LOU064 resulted in full BTK occupancy in blood and spleen of female rats. When determining the duration of BTK occupancy in rat after a single oral dose of LOU064 BTK occupancy half-life was ˜87 hours in blood but only ˜5 hours in spleen. Without wishing to be bound by any theory, it has been hypothesized that the longer persistence of BTK occupancy in blood may reflect that BTK expressing cells in peripheral blood are resting and metabolically relatively inactive compared to tissues such as spleen, lymph nodes and lung.

BTK occupancy in different tissues is relevant to efficacy and optimum dosage selection in different indications. However, there is no currently agreed complete picture of all tissues that are relevant to the multiple sclerosis indication and therefore which tissue(s) need to be penetrated for treating MS. As reported in rat, the BTK occupancy and BTK occupancy half-life is different in blood and in various tissues.

BTK occupancy half-life is dependent on the turnover rates (ability of the BTK cells to regenerate). Such turnover rates differ in each tissue and are species specific. The BTK occupancy is further dependent on the PK/PD properties of a compound which is also species dependent.

It was discovered that the BTK occupancy in blood may not be fully correlated for the MS indication.

BTK occupancy in other tissues such as spleen, lymph nodes and lung may be more correlated to efficacy in the MS indication than the BTK occupancy in blood. In addition, BTK occupancy in brain may be another relevant factor for efficacy in the treatment of MS.

BTK occupancy in brain depends on several factors. Some factors are compound specific. For example, one factor is the brain blood barrier permeability of a compound, its affinity to P-glyco protein transporter controlling the degree of efflux pump out of the brain.

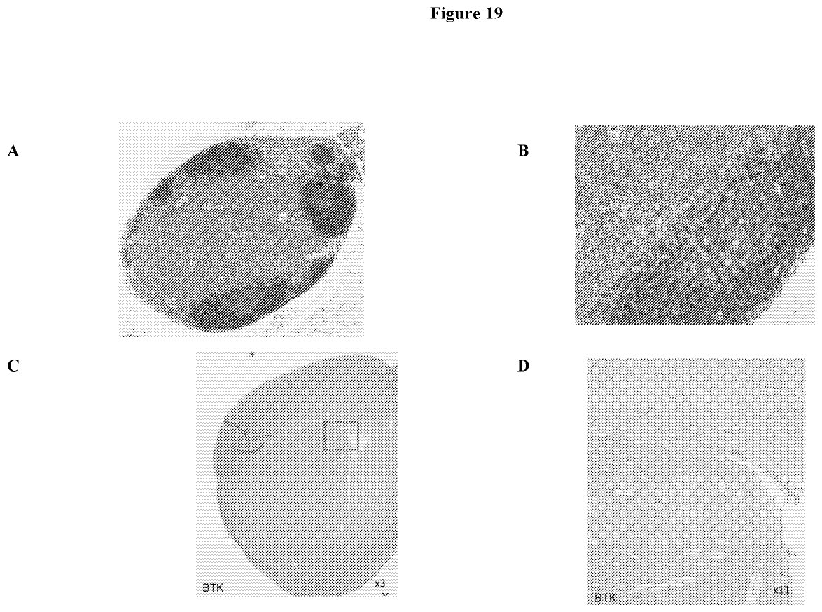

Another factor is the uncertainty concerning the expression of BTK in brain, since in naive mouse brain, in particular in the corpus callosum, BTK could not be detected (FIG. 19).

There is therefore a high level of unpredictability in order to determine the efficacious dose of a BTK inhibitor for the treatment of MS.

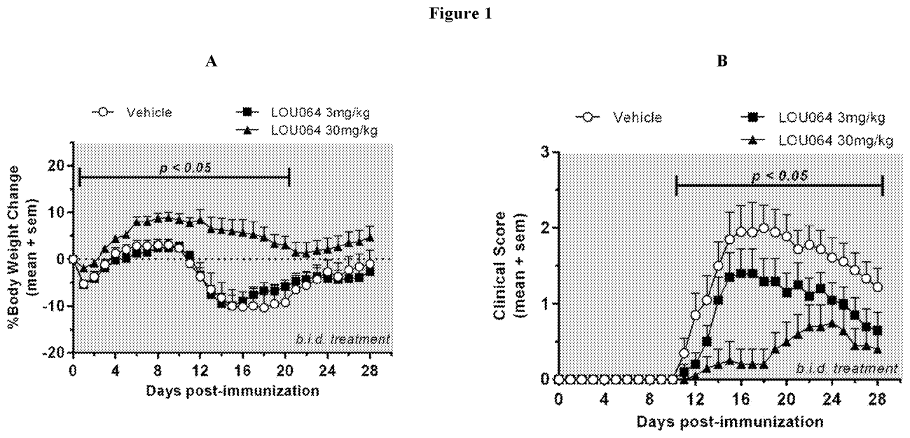

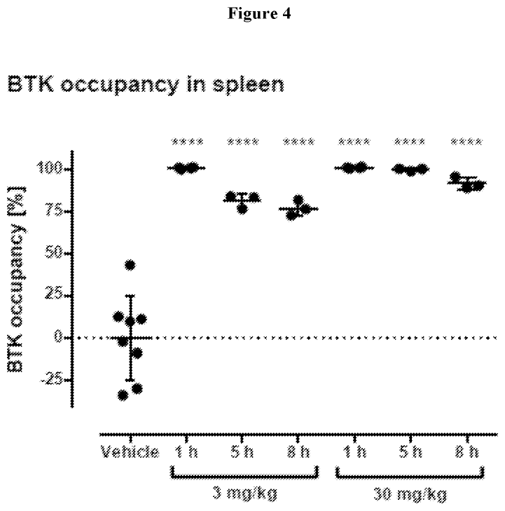

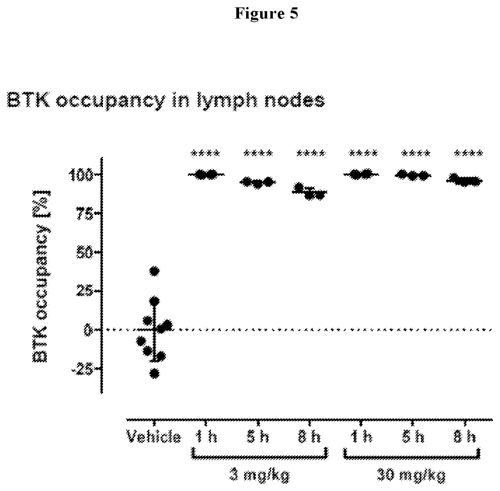

In order to assess in vivo efficacy of LOU064, the compound was tested in the mouse Experimental Autoimmune Encephalitis (EAE) model. At a dose of 3 mg/kg b.i.d. of LOU064 complete BTK occupancy in spleen and lymph nodes was observed 1 h after dosing (FIGS. 4 and 5). However, this dose of 3 mg/kg b.i.d. of LOU064 led to only a fairly weak reduction of neurologic symptoms as well as EAE-induced weight loss (FIG. 1) even though complete BTK occupancy in spleen and lymph nodes has been reached at this dose.

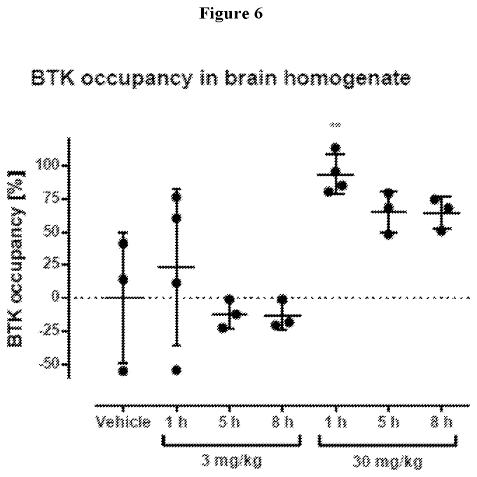

Therefore, BTK occupancy was also assessed in brain homogenates that had been prepared for compound exposure analysis (FIG. 6). In contrast to what was observed in spleen and lymph nodes the 3 mg/kg dose surprisingly led to only minimal BTK occupancy in brain homogenates at the 1 hour timepoint. However, even more surprisingly, the dose group receiving 30 mg/kg b.i.d. LOU064 showed maximal BTK occupancy.

Considering that full BTK occupancy in the brain requires a higher dose (30 mg/kg b.i.d.), it was found according to this invention that LOU064 at a dose of 30 mg/kg b.i.d. strongly reduced neurologic symptoms, as well as EAE-induced weight loss in mice.

Additionally, BTK occupancy in spleen determined at 1, 5 and 8 hours after b.i.d. oral dosing of LOU064 (FIG. 4) also shows a more sustained occupancy after the 30 mg/kg dose.

Without being bound to theory, it was concluded that BTK occupancy in brain may be more relevant for the treatment of MS. This was completely unexpected since in naive mouse brain, in particular in the corpus callosum, BTK could not be detected (FIG. 19).

Based on a conversion model between animals and human (Journal of basic and clinical pharmacy, 7(2), 27-31), the calculated human equivalent dose (HED) for 30 mg/kg b.i.d. corresponds to˜170 mg for a 70 kg person (Nair, A. B., & Jacob, S. (2016).

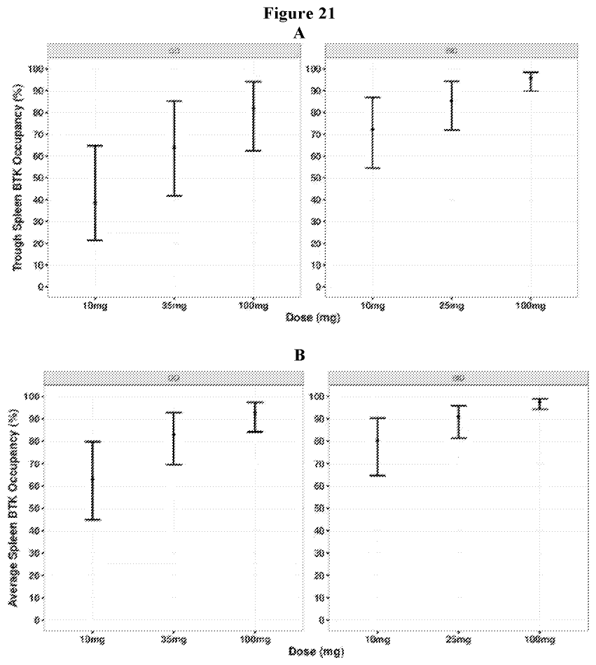

Finally, according to a prediction model of BTK occupancy in human, a b.i.d. dosing was shown to be more effective than QD dosing at the same dose to achieve higher BTK occupancy (FIG. 21). Accordingly, a dose of LOU064 of 100 mg b.i.d. was chosen for clinical studies.

Particularly surprisingly, LOU064 exhibits superior efficacy and safety in the treatment of multiple sclerosis already when it is administered orally at a dose of 100 mg twice daily.

Hence, a subject of the present invention relates to LOU064 or a pharmaceutically acceptable salt thereof for use in the treatment of multiple sclerosis.

Generally, the present invention concerns the treatment of multiple sclerosis. In a preferred embodiment of the invention LOU064 is administered for the treatment of relapsing forms of multiple sclerosis (RMS) including relapsing-remitting multiple sclerosis (RRMS), secondary progressive multiple sclerosis (SPMS), in particular active SPMS, and clinically isolated syndrome (CIS), preferably in adults.

In particular, the present invention relates to the treatment of relapsing-remitting multiple sclerosis (RRMS). Alternatively, the present invention relates to the treatment of secondary progressive MS (SPMS), in particular active SPMS. Further alternatively, the present invention relates to the treatment of clinically isolated syndrome (CIS).

In one embodiment LOU064 is administered orally at a dose of about 10 mg to about 500 mg twice daily or at a dose of about 25 mg to about 400 mg twice daily or at a dose of about 50 mg to about 300 mg twice daily, or at a dose of about 50 mg to about 250 mg twice daily, or at a dose of about 50 mg to about 150 mg twice daily.

Preferably LOU064 is administered orally at a dose of about 50 mg to about 150 mg twice daily, more preferably at a dose of about 100 mg twice daily.

In another embodiment, LOU064 is administered orally at a dose of about 100 mg to about 300 mg twice daily, more preferably at a dose of about 250 mg twice daily.

Within a suitable pharmaceutical formulation, LOU064 may be present in any pharmaceutically acceptable form. It may be preferable to include LOU064 in the pharmaceutical formulation as nanosized or as microsized particles.

If LOU064 is present in the pharmaceutical formulation in the form of nanosized particles, the mean particle size can be less than 1000 nm. Preferably, the mean particle size of LOU064 can be less than 500 nm, more preferably less than 250 nm.

In a preferred embodiment, the mean particle size of LOU064 can be between about 50 nm and about 1000 nm, or between about 50 nm and about 750 nm, or between about 60 nm and about 500 nm, or between about 70 nm and about 350 nm, or between about 100 nm and about 170 nm, More preferably, the mean particle size of LOU064 may be between about 100 nm and about 350 nm, or between about 110 nm and about 200 nm, or between about 120 nm and about 180 nm or between about 120 nm and about 160 nm, preferably the mean particle size of LOU064 can be about 150 nm to about 200 nm.

If LOU064 is present in the pharmaceutical formulation in the form of nanosized particles, oral administration is preferably at a dose of about 50 mg to about 150 mg twice daily, more preferably at a dose of about 100 mg twice daily.

If LOU064 is present in the pharmaceutical formulation in the form of microsized particles, the mean particle size can be 1-5 μm or preferably 1.0-1.5 μm. Preferably, the mean particle size of LOU064 can be 1.1 to 1.3 μm.

If LOU064 is present in the pharmaceutical formulation in the form of microsized particles, oral administration is preferably at a dose of about 100 mg to about 300 mg twice daily, for example at a dose of about 100 mg twice daily.

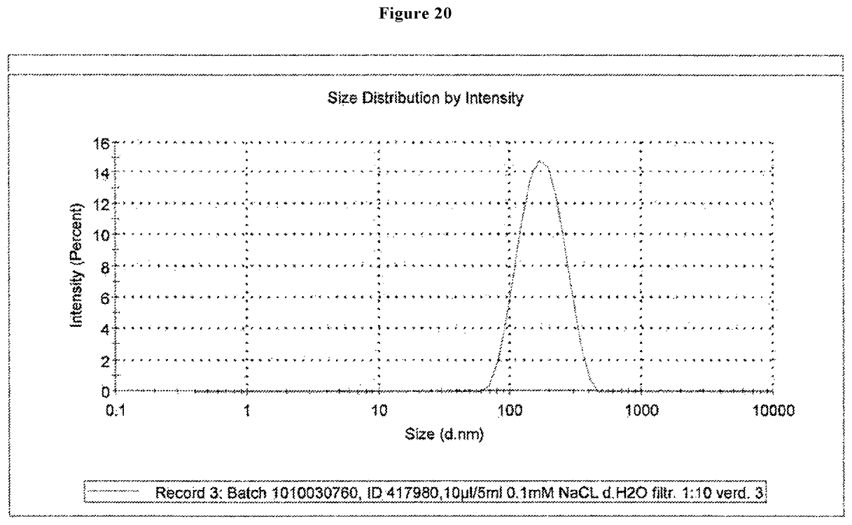

In a preferred embodiment the polydispersity index (PI) is between 0.01 and 0.5, more preferably between 0.1 and 0.2, in particular 0.12-0.14. A preferred particle size distribution is shown in FIG. 20.

The above-mentioned mean particle sizes are intensity weighted. The mean particle size can be determined by means of dynamic light scattering. Preferably, the mean particle size is determined by Photon Correlation Spectroscopy (PCS). In particular, for determining the mean particle size the device “Zetasizer Nano ZS”, Version 7.13 from Malvern Panalytical Ltd., UK can be used.

Preferably, the measurement is carried out as wet dispersion method using 0.1 mM NaCl solution in purified water (1:10), wherein the attenuator index is 2-9, in particular 5. The measurement is preferably carried out at 25° C. Further preferred settings of the measurement systems are as follows:
Cell: Disposable sizing cuvette
Count Rate (kcPs): 315
Duration: 60 sec
Measurement Position (mm): 4.65.

In one embodiment of the invention, a LOU064 composition is formulated in accordance with routine procedures as a pharmaceutical composition adapted for oral administration to human beings. Typically, compositions for oral administration are capsules or tablets.

In one embodiment, a formulation for LOU064 can be formulated according to a formulation disclosed in U.S. application No. 63/141,558 or its family members, herein incorporated by reference.

According to the invention, a suitable pharmaceutical composition for oral administration comprises LOU064 and binder.

Suitable binders include polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, or a mixture thereof. Preferably the binder is polyvinylpyrrolidone-vinyl acetate copolymer.

The weight ratio of LOU064 and binder can be from about 3:1 to about 1:3; e.g. about 3:1, about 2:1, about 1:1, preferably the weight ratio of LOU064 and binder is about 2:1 or about 1:1.

Preferably, a suitable pharmaceutical composition for oral administration comprises LOU064, binder and surfactant.

Suitable surfactants include sodium lauryl sulfate, potassium lauryl sulfate, ammonium lauryl sulfate, sodium lauryl ether sulfate, polysorbates, perfluorobutanesulfonate, dioctyl sulfosuccinate, or a mixture thereof. Preferably the surfactant is sodium lauryl sulfate.

The weight ratio of LOU064, binder and surfactant is about 2:1:0.5, or about 2:1:0.1, or about 2:1:0.08, or about 2:1:0.05, or about 2:1:0.04, or about 2:1:0.03, or about 2:1:0.02. Preferably, the weight ratio of LOU064, binder and surfactant is about 2:1:0.08 or about 1:1:0.05.

In a particularly preferred embodiment, a suitable pharmaceutical composition for oral administration comprises LOU064, binder and surfactant, wherein the binder is polyvinylpyrrolidone-vinyl acetate copolymer (copovidone) and the surfactant is sodium lauryl sulfate (SLS), and wherein the weight ratio of LOU064, copovidone and SLS is about 2:1:0.08. It is further particularly preferred that LOU064 is present in this pharmaceutical composition in the form of nanosized particles, preferably having a mean particle size as measured by PCS of between about 100 nm and about 200 nm

If a dose of LOU064 is missed, it should preferably be administered as soon as possible without waiting until the next scheduled dose. Subsequent doses should be administered at the recommended intervals.

Short-term safety of LOU064 at single doses up to 600 mg and further at 100 mg b.i.d. for up to 18 days has been shown in Phase I clinical studies. However, no data concerning long-term safety was available.

Considering dose-limiting side effects observed with the covalent irreversible BTK inhibitors evobrutinib and tolebrutinib, with evobrutinib showing dose-limiting liver enzyme elevations already at a dose of 75 mg b.i.d. in Phase II clinical studies and tolebrutinib showing dose-limiting diarrhea (Becker A. et al., 2019, Clin Transl Sci; 13,325-336; Montalban X. et al., 2019, N Engl J Med; 380(25): 2406-17, Smith P. F. et al., 2019, ACTRIMS Forum, Feb. 28, 2019, P072), it is very surprising that LOU064 at an even higher dose of 100 mg b.i.d. over an extended period of time does not induce any such adverse effects, rendering LOU064 particularly suitable for long-term treatment. In particular, LOU064 does not induce any dose-limiting liver enzyme elevations and other off-target-effects at a dose of 100 mg b.i.d. over an extended period of time.

In particular, it was surprisingly found that LOU064 not only prevents unwanted side-effects for longer than other DMTs, particularly than other BTK inhibitors, but also preserves activity, i.e. maintains efficacy for longer than other DMTs, particularly than other BTK inhibitors.

It was further surprisingly found that LOU064 is delaying the worsening of symptoms for longer than other DMTs, particularly than other BTK inhibitors.

Hence, in a preferred embodiment of the present invention, LOU064 or a pharmaceutically acceptable salt thereof for use in treating MS is used in a long-term treatment. The term long-term treatment indicates that LOU064 or a pharmaceutically acceptable salt thereof is used over an extended period of time. For example, LOU064 or a pharmaceutically acceptable salt thereof can be used for more than 2 years, 3 years, 4 years, 5, years, 10 years. LOU064 or a pharmaceutically acceptable salt thereof might be used up to 5 years, 10 years, 15 years, 20 years or for life.

Because the treatment of multiple sclerosis may span decades, the need often arises to make changes to the treatment plan in order to accommodate changing circumstances such as adverse effects, treatment failure, breakthrough disease, disease progression, comorbidities, life cycle events such as pregnancy and lactation, and/or evolving patient preferences.

Switching drugs, or the discontinuation of immunomodulatory agents altogether, may leave patients vulnerable to relapse or disease progression. In some cases, severe MS disease activity is noted clinically and on MRI after treatment withdrawal. When this disease activity is disproportionate to the pattern observed prior to treatment initiation, patients are said to have experienced rebound.

According to the invention, LOU064 provides a powerful and efficacious treatment strategy to prevent relapses. LOU064 further provides a powerful and efficacious treatment strategy for breakthrough disease with other DMTs. Moreover, LOU064 provides a powerful and efficacious treatment strategy for prevention of rebound after cessation of another DMT.

Hence, in one embodiment LOU064 is used in patients who had been treated with a disease-modifying therapy other than LOU064. In other words, patients who had been treated with an earlier disease-modifying therapy other than LOU064 are switched from the earlier disease-modifying treatment to LOU064.

In one embodiment the drug of the earlier disease-modifying therapy other than LOU064 is selected from a B cell and/or T cell inhibitor, teriflunomide, mitoxantrone, dimethyl fumarate, cladribine, fingolimod, siponimod, ponesimod, glatiramer acetate and beta interferon.

In a preferred embodiment, LOU064 is administered to patients who discontinued earlier DMT, e.g. anti-CD20 therapy, because of side effects such as severe infusion-related reactions or recurrent infections.

In a preferred embodiment the patient is switched from an earlier disease-modifying therapy to LOU064 in cases where the earlier disease-modifying therapy lacks efficacy. A lack of efficacy is present, for example, if a patient who is on a disease-modifying therapy (DMT) shows signs of disease activity, such as relapses or lesions. Lack of efficacy can be defined as not stopping or not appropriately slowing down disease progression. In other words, the present invention is directed to use of LOU064 for treating non-responders to the earlier DMT.

In another preferred embodiment the patient is switched from an earlier disease-modifying therapy to LOU064 in cases where the patient lacks tolerability for the earlier disease-modifying therapy. Preferably, a lack of tolerability relates to the presence of adverse events such as headache, dizziness, nausea, infections (such herpes zoster), macular edema, infusion-related reactions or recurrent infections. In a preferred embodiment of the invention, the earlier disease-modifying therapy other than LOU064 is discontinued before initiation of LOU064 administration.

Although in general LOU064 and another disease-modifying therapy may be pursued at the same time, it is preferred that LOU064 treatment is a monotherapy, i.e. LOU064 is preferably the only disease-modifying drug that is administered.

Thus, in one embodiment LOU064 is for use in the treatment of multiple sclerosis, wherein the treatment is a monotherapy.

LOU064 is anticipated to be a sensitive CYP3A substrate and it cannot be ruled out that its oral drug exposure may be increased several fold when administered with strong CYP3A4 inhibitors. Likewise, strong inducers of CYP3A4 may significantly decrease the exposure and lead to reduced efficacy. These properties of LOU064 are not only relevant for MS but also for any BTK-induced condition.

Concomitant administration with strong CYP3A4 inhibitors and/or inducers may possibly cause substantial changes in LOU064 drug exposure, and should be avoided. CYP3A4 inhibitors include strong CYP3A4 inhibitors such as boceprevir, clarithromycin, cobicistat, conivaptan, danoprevir/ritonavir, darunavir/ritonavir, elvitegravir/ritonavir, grapefruit juice, idelalisib, indinavir, indinavir/ritonavir, itraconazole, ketoconazole, LCL161, lopinavir/ritonavir, mibefradil, nefazodone, nelfinavir, posaconazole, ritonavir, saquinavir, saquinavir/ritonavir, telaprevir, telithromycin, tipranavir/ritonavir, troleandomycin, Viekira pack or/and voriconazole.

Therefore, in another preferred embodiment LOU064 is not administered concomitantly with a strong inhibitor and/or inducer of CYP3A4.

It has further been found that LOU064 can be co-administered with oral contraceptives such as ethinylestradiol or levonorgestrel without a major impact on their exposure and efficacy. Therefore, in a preferred embodiment, LOU064 is co-administered with oral contraceptives.

In a preferred embodiment, no premedication is administered prior to the first dose of LOU064.

As a covalent irreversible BTK inhibitor LOU064 acts via irreversible inhibition of BTK which is countered by de novo protein synthesis. Thus, without wishing to be bound by any theory, it is believed that, whereas reconstitution of the B cell pool after B cell depletion can take several months, the restoration of B cell function after BTK inhibition can be achieved shortly after discontinuation, in particular within days. Therefore, if need be, this therapy could rapidly be stopped which provides clinicians and patients with easier and faster reaction capacity when unforeseen circumstances arise.

Thus, in one embodiment LOU064 is advantageously selected if the patient is planning to get pregnant within the next 12 months.

In another embodiment LOU064 is advantageously selected if the patient will undergo chemotherapy within the next 12 months.

Especially in light of the 2020 COVID-19 pandemic, B cell-depleted patients have a higher risk of infection. Furthermore, the absence of a fully functional adaptive immune response likely leads to a more severe course.

However, since LOU064 does not result in depletion of the pool of B cells, cessation of the therapy leads to a quick restoration of complete B cell function. This gives patients and treating physicians the possibility to quickly respond to infectious disease or vaccination requirements, in particular vaccination with live vaccines and attenuated vaccines.

According to the invention, LOU064 can be administered during an infection, e.g. during a COVID-19 infection. Thus, LOU064 administration can be continued during an infection, e.g. during a COVID-19 infection.

Alternatively, LOU064 administration is delayed in patients with an active infection, e.g. COVID-19, until the infection is resolved.

Thus, one embodiment of the invention relates to LOU064 for use in the treatment of multiple sclerosis, wherein a patient acutely or previously infected by COVID-19 is treated.

In a preferred embodiment LOU064 treatment is continued during COVID-19 infection.

In a further embodiment LOU064 treatment is interrupted during COVID-19 infection and continued after overcoming the infection.

A still further embodiment of the invention relates to LOU064 for use in the treatment of a BTK-mediated condition, particularly multiple sclerosis, wherein the patient is vaccinated during LOU064 therapy. Alternatively, the patient can be vaccinated during LOU064 therapy with non-live vaccines.

In one embodiment, the patient is vaccinated with quadrivalent Influenza vaccine, the PPV-23 vaccine or the KLH neoantigen vaccine during LOU064 therapy (e.g. at day 15 after initiating LOU064 therapy). In one aspect of this embodiment, the patient receiving quadrivalent Influenza vaccine, achieves a response as defined by a >4-fold increase of anti-hemmaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination.

Another embodiment of the invention relates to LOU064 for use in the treatment of a BTK-mediated condition, particularly multiple sclerosis, wherein LOU064 treatment is discontinued for vaccination, in particular wherein LOU064 treatment is discontinued 5-10 days (e.g. 7 or 8 days), preferably 6 weeks prior to vaccination and continued after vaccination, e.g. 5-20 days, preferably 5-10 days or most preferably 10-15 days after vaccination. In a particular aspect of this embodiment, the patient is vaccinated with quadrivalent Influenza vaccine, the PPV-23 vaccine or the KLH neoantigen vaccine after discontinuing LOU064 treatment (e.g. 5-10 days or 7 or 8 days after discontinuing LOU064 treatment). In one aspect of this embodiment, the patient receiving quadrivalent Influenza vaccine, achieves a response as defined by a >4-fold increase of anti-hemmaglutinin antibody titers at 28 days after vaccination compared to baseline. In another aspect of this embodiment, the patient receiving the PPV-23 vaccine achieves a >2-fold increase of IgG titers 28 days after vaccination compared to baseline. In yet another embodiment, the patient receiving the KLH neoantigen vaccine achieves a T-cell dependent antibody response as measured by anti-KLH IgG and IgM titers 28 days after vaccination. LOU064 treatment is then continued starting on Day 29 after vaccination.

In an alternative embodiment the vaccination is a vaccination with live vaccines and/or attenuated vaccines. In a preferred embodiment, LOU064 can be administered irrespective of body weight, sex, age, race or baseline B-cell count.

For example, it is preferred that a 35-year-old woman having a body weight of 60 kg receives the same dose as a 50-year old man having a body weight of 90 kg. In particular, body weight, sex, age, race or baseline B-cell count do not have a clinically meaningful effect on the pharmacokinetics of LOU064.

According to the invention LOU064 treatment is equally effective in any racial or ethnic groups.

Therefore, the invention further relates to LOU064 for use in the treatment of multiple sclerosis, wherein the treatment is an ethnic insensitive treatment.

In one embodiment of the invention the patient receiving LOU064 or a pharmaceutically acceptable salt thereof for the treatment of MS is selected according to the following criteria:
the patient has an EDSS score of 0 to 5.5 prior to the first administration of LOU064,
the patient has experienced at least one relapse during the previous year or two relapses during the previous two years prior to the first administration of LOU064, and
the patient had a positive Gd-enhancing MRI scan during the previous year/6 months prior to the first administration of LOU064, In a preferred embodiment of the invention, LOU064 is administered after a relapse.

In another preferred embodiment of the invention, LOU064 is administered after the detection of at least one Gd+ lesion in the previous 12 month prior to first administration of LOU064.

In still another preferred embodiment of the invention, LOU064 is administered after the detection of new or enlarging T2 lesions.

In another embodiment of the invention LOU064 is selected for use in the treatment of multiple sclerosis in patients for which SiP modulator treatment would not be of choice due to a less favourable risk/benefit ratio. Such patients are for example patients susceptible to or suffering from one or more disease or disorders affecting the heart or heart rhythm, respiratory functions, eyes, hepatic functions. In particular, such patients include patients who could be more susceptible to adverse events such as a transient reduction of heart rate and cardiac conduction.

In the present invention, it was unexpectedly found that administration of LOU064 advantageously leads to at least one of the following:
a reduced mean total number of gadolinium-enhancing lesions as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon.
a reduced annualized relapse rate as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or inteferon,
a longer time to reach 3-month confirmed disability progression as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon.

In this regard a further subject of the present invention is LOU064 for use in the treatment of relapsing multiple sclerosis, wherein LOU064 reduces the mean total number of gadolinium-enhancing lesions as compared to untreated patients and/or as compared to patients receiving another disease-modifying treatment selected from interferon, teriflunomide, glatiramer acetate, dimethyl fumarate, preferably interferon, teriflunomide and dimethyl fumarate, more preferably teriflunomide or interferon, within up to 60 months, or within up to 30 months or within up to 24 months, preferably 12-24 months.

Still a further subject of the present invention is LOU064 for use in the treatment of relapsing multiple sclerosis, wherein LOU064 leads to a reduced annualized relapse rate as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to a longer time to reach 3-month confirmed disability progression as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an improvement of MS symptoms as measured by a decrease in scores in one or more patient-reported outcome (PRO) (i.e. MSIS-29, PHQ-9, GAD-7, FSIQ-RMS and BPI-SF) as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an improvement of MS symptoms as measured by a decrease in scores in one or more patient-reported outcomes (PROs) (i.e. MSIS-29, PHQ-9, GAD-7, FSIQ-RMS and BPI-SF) as compared to baseline within up to 60 months, or within up to 30 months, or within up to 24 months, preferably 12-24 months. In one aspect of this embodiment, the improvement of MS symptoms is achieved by a decrease of at least 6 points (preferably at least 8 points) as compared to baseline in the MSIS-29 score after treatment with LOU064 (100 mg bid) within up to 60 months, or within up to 30 months, or within up to 24 months, preferably within 12-24 months. In another aspect of this embodiment, the improvement of MS symptoms is achieved by a decrease of at least 2 points (preferably at least 3 points, more preferably at least 5 points) in the PHQ9 score as compared to baseline after treatment with LOU064 (100 mg bid) within up to 60 months, or within up to 30 months, or within up to 24 months, preferably within 12-24 months. In yet another aspect of this embodiment, the improvement of MS symptoms is achieved by a decrease of at least 2 points (preferably at least 3 points) in the GAD-7 score as compared to baseline after treatment with LOU064 (100 mg bid) within up to 60 months, or within up to 30 months, or within up to 24 months, preferably within 12-24 months. In yet another aspect of this embodiment, the improvement of MS symptoms is achieved by a decrease of at least 1 point (preferably at least 2 points) in the BPI-SF score as compared to baseline after treatment with LOU064 (100 mg bid) within up to 60 months, or within up to 30 months, or within up to 24 months, preferably within 12-24 months.

Another subject of the present invention is LOU064 for use in the treatment of relapsing multiple sclerosis, wherein LOU064 leads to an improvement of MS symptoms as measured by an increase in scores in the HUI-III patient-reported outcome (PRO) as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an improvement of MS symptoms as measured by an increase in scores in the HUI-III patient-reported outcomes (PROs) as compared to baseline within up to 60 months, or within up to 30 months, or within up to 24 months, preferably 12-24 months. In one aspect of this embodiment, the improvement of MS symptoms is achieved by an increase of at least 0.02 points (preferably at least 0.03 points) in the HUI score after treatment with LOU064 (100 mg bid) within up to 60 months, or within up to 30 months, or within up to 24 months, preferably within 12-24 months.

Another subject of the present invention is LOU064 for use in the treatment of relapsing multiple sclerosis, wherein LOU064 leads to an improvement of MS symptoms as measured by a decrease in SDMT scores as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an improvement of MS symptoms as measured by a decrease in SDMT as compared to baseline within up to 60 months, or within up to 30 months, or within up to 24 months, preferably 12-24 months. In one aspect of this embodiment, the improvement of MS symptoms is achieved by a decrease of at least 3 points (preferably at least 5 points) in the SDMT score after treatment with LOU064 (100 mg bid) within up to 60 months, or within up to 30 months, or within up to 24 months, preferably within 12-24 months.

Another subject of the present invention is LOU064 for use in the treatment of relapsing multiple sclerosis, wherein LOU064 leads to an increase in walking speed as measured by T25 FW as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an increase in walking speed as measured by T25 FW as compared to baseline within up to 60 months, or within up to 30 months, or within up to 24 months, preferably 12-24 months. The baseline T25 FW-result is defined as the last assessment prior to the first dose of LOU064.

Another subject of the present invention is LOU064 for use in the treatment of relapsing multiple sclerosis, wherein LOU064 leads to an increase in the speed with which the patient perform the 9HPT test as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon, within up to 60 months, or within up to 30 months, or within up to 24 months, preferably 12-24 months.

Another subject of the present invention is LOU064 for use in the treatment of relapsing multiple sclerosis, wherein LOU064 leads to an increase in the speed with which the patient performs the 9HPT test as compared to baseline within up to 60 months, or within up to 30 months, or within up to 24 months, preferably 12-24 months. The baseline 9HPT-result is defined as the last assessment prior to the first dose of LOU064.

In the present invention, it was further unexpectedly found that upon administration of LOU064 by week 12 or by week 24 of treatment the levels of alanine aminotransferase (ALT), aspartate aminotransferase (AST), and lipase advantageously do not change by more than 10% as compared to the baseline level at the start of therapy.

According to the present invention it was unexpectedly found that administration of LOU064 advantageously leads to at least one of the following:
a relative reduction of the annualized relapse rate of 45-50%, preferably 55-60%, as compared to teriflunomide,
a relative reduction of the mean total number of gadolinium-enhancing lesions (Gd+T1) of 60-75%, preferably 90-95%, as compared to teriflunomide,
a relative reduction of new/enlarging T2 lesions of 65-70%, preferably 80-85%, as compared to teriflunomide,
a relative reduction of the time to reach 3-month confirmed disability progression (3mCDP) of ˜30%, preferably 30-35%, as compared to teriflunomide,
a relative reduction of the time to reach 6-month confirmed disability progression (6mCDP)of ˜30-35%, preferably 35-40%, as compared to teriflunomide,
no evidence of disease ability of up to 6-7/10 patients, preferably of up to 8-9/10 patients at 12 to 60 months, or at 12 to 30 months or at 12-24 months (NEDA-3),
a relative reduction of serum Nfl concentration of at least 20% as compared to teriflunomide.

It was further unexpectedly found that LOU064 treatment is as effective in reducing the annual relapse rate as a CD20-depleting therapy, in particular superior in reducing the annual relapse rate as compared to a CD20-depleting therapy.

The invention further relates to LOU064 for producing a medicament for use in the treatment of multiple sclerosis, wherein preferably the medicament is administered orally at a dose of about 50 mg to about 150 mg twice daily.

A further subject of the invention is a method for treating multiple sclerosis, said treatment comprising oral administration of LOU064 to a patient in need of such treatment, preferably at a dose of about 50 mg to about 150 mg twice daily.

A further subject of the present invention is a method for the manufacture of a medicament for use in the treatments described above.

### DETAILED DESCRIPTION OF THE INVENTION

### Definitions

As used herein, a “BTK inhibitor” is any substance capable of inhibiting Bruton's tyrosine kinase (BTK), which is a cytoplasmic tyrosine kinase and member of the TEC kinase family. BTK is selectively expressed in cells of the adaptive and innate immune system including B cells, macrophages, mast cells, basophils, and also in thrombocytes. Examples of BTK inhibitors include non-covalent, reversible BTK inhibitors such as fenebrutinib as well as covalent, irreversible inhibitors of BTK such as evobrutinib, tolebrutinib, rilzabrutinib, tirabrutinib, branebrutinib, orelabrutinib and remibrutinib (LOU064).

As used herein, “LOU064”, which has the INN remibrutinib, refers to the compound N-(3-(6-amino-5-(2-(N-methylacrylamido)ethoxy)pyrimidin-4-yl)-5-fluoro-2-methylphenyl)-4-cyclopropyl-2-fluorobenzamide having the following structural formula:

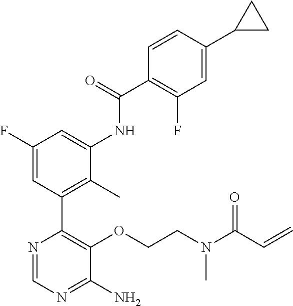 [SMILES: C=CC(=O)N(C)CCOc1c(N)ncnc1-c1cc(F)cc(NC(=O)c2ccc(C3CC3)cc2F)c1C]

or a pharmaceutically acceptable salt thereof, or a free form thereof. In one embodiment, LOU064 refers to a crystalline form of LOU064 as disclosed in example 1 of WO2020/234779, which is herein incorporated by reference.

LOU064 is a selective potent irreversible covalent BTK inhibitor and is among a new generation of designed covalent enzyme inhibitors (Angst et al 2020). The compound was first disclosed in example 6 of WO 2015/079417, filed on Nov. 28, 2014.

The terms “treatment” or “treat” can be defined as the application or administration of e.g. LOU064 to a patient, where the purpose is to abolish, reduce or alleviate the symptoms of a disease such as multiple sclerosis (MS). In particular, the term “treatment” comprises the achievement of a clinically meaningful effect for the patient, for example the achievement of a clinically meaningful reduction of the annual relapse rate when treating RMS. The term may further include delaying progression to a progressive form of MS.

The term “patient” as used herein can be a mammal, e.g. a primate, preferably a higher primate, especially preferred a human (e.g. a patient having a risk or at risk of having a disorder described herein). Preferably, the patient is an adult. Theoretically, also geriatric patients are included, however, patients between 18 and 60 years of age are preferred.

As used herein, a patient can be “in need of” a treatment if such a patient would benefit medically or in terms of the quality of life from such treatment.

As used herein, the term “administering” or “administration” of LOU064 can mean providing LOU064 to a patient in need of treatment. Administration “in combination with” one or more further therapeutic agents includes simultaneous (concurrent) and consecutive administration in any order and in any route of administration.

As used herein, a “therapeutically effective amount/dose” can refer to an amount/dose of LOU064 that is effective, i.e. achieves a clinically meaningful effect.

The term “adverse event” (AE) can relate to any untoward medical occurrence in a patient or clinical investigation wherein the subject is administered a pharmaceutical product which does not necessarily have a causal relationship with this treatment. An adverse event (AE) can therefore be any unfavorable and unintended sign (including an abnormal laboratory finding), symptom or disease temporally associated with the use of a medicinal (investigational) product, whether or not related to the medicinal (investigational) product.

The phrases “therapeutic regimen” or “treatment regimen” can mean the regimen used to treat an illness or to prevent a disease condition or the development of a disease, e.g. the dosing used. A therapeutic regimen or treatment regimen may include an induction regimen, a loading regimen and a maintenance regimen (e.g. a loading dose as an initial dose of a drug, preferably an initial higher dose, that may be given at the beginning of a course of treatment (e.g. a DMT) before succeeding with a maintenance dose, preferably dropping down to a lower maintenance dose).

As used herein, “multiple sclerosis” refers to any form of the disease including primary progressive multiple sclerosis (PPMS) and relapsing multiple sclerosis (RMS) which encompasses relapsing-remitting multiple sclerosis (RRMS), secondary progressive multiple sclerosis (SPMS), in particular active SPMS (with an occasional relapse and/or evidence of new MRI activity), and clinically isolated syndrome (CIS).

Primary progressive MS (PPMS) is characterized by worsening neurologic function (accumulation of disability) from the onset of symptoms, without early relapses or remissions. PPMS can be further characterized at different points in time as either active (with an occasional relapse and/or evidence of new MRI activity) or not active, as well as with progression (evidence of disease worsening on an objective measure of change over time, with or without relapse or new MRI activity) or without progression. References is made to Lublin 2014.

Each person's experience with PPMS will be unique. PPMS can have brief periods when the disease is stable, with or without a relapse or new MRI activity, as well as periods when increasing disability occurs with or without new relapses or lesions on MRI.

The term RMS (relapsing multiple sclerosis) encompasses RRMS, SPMS, in particular active SPMS, and CIS.

Relapsing-remitting multiple sclerosis (RRMS) is characterized by relapses, e.g. defined as a new neurologic deficit or episode of neurologic worsening lasting longer than 24 h, often in the absence of fever or infection.

There is no apparent progression of the disease during the periods of remission. At different points in time, RRMS can be further characterized as either active (with relapses and/or evidence of new MRI activity) or not active, as well as worsening (a confirmed increase in disability over a specified period of time following a relapse) or not worsening. Reference is made to Lublin 2014, Neurology: 2014 Jul 15; 83(3): 278-286.

Secondary progressive multiple sclerosis (SPMS) follows an initial relapsing-remitting course. Most people who are diagnosed with RRMS will eventually transition to a secondary progressive course in which there is a progressive worsening of neurologic function (accumulation of disability) over time. SPMS can be further characterized at different points in time as either active (with relapses and/or evidence of new MRI activity) or not active, as well as with progression (evidence of disease worsening on an objective measure of change over time, with or without relapses) or without progression. Reference is made to Lublin 2014, Neurology: 2014 Jul 15; 83(3): 278-286.

Each person's experience with SPMS will be unique. SPMS follows after relapsing-remitting MS. Disability gradually increases over time, with or without evidence of disease activity (relapses or changes on MRI). In SPMS, occasional relapses may occur, as well as periods of stability. According to the present invention, the term SPMS particularly refers to active SPMS, i.e. SPMS with evidence of disease activity as determined by the presence of relapses and/or changes on MRI.

Clinically isolated syndrome (CIS) may refer to a single clinical attack of central nervous system (CNS) inflammatory demyelinating symptoms that are suggestive of multiple sclerosis (MS). CIS presentations can be monofocal or multifocal and typically may involve the optic nerve, brainstem, cerebellum, spinal cord or cerebral hemispheres. Reference is made to Miller et al, Clinically isolated syndromes, Lancet Neurol. 2012; 11:157-169.

Relapses can be defined as a new neurologic deficit or episode of neurologic worsening, preferably lasting longer than 24 h. In other words, relapses can be regarded as discrete episodes (in the art also referred to as “attacks”, “flare-ups” or “exacerbations”) of neurologic dysfunction, preferably lasting at least 24 h. Usually, relapses are followed by full or partial recovery and a period in which there is no symptom progression or accumulation of disability (remission).

Relapses are presumed to be caused by a new or enlarging demyelinating plaque at the site of an inflammatory event within the central nervous system (CNS).

Revised McDonald criteria (Thompson et al 2018)

Under the revised McDonald Criteria, an MS diagnosis is likely if myelin damage is disseminated in space (DIS), as seen in an MRI as:
At least one T2 bright lesion in at least two or four CNS locations: the juxtacortical, perventricular and infratentorial areas of the brain, and the spinal cord (T2 is the most common MRI scan used to diagnose MS and to detect areas of myelin damage, old and new, in the brain and spinal cord).
These lesions need not be gadolinium enhanced (contrast material).

Regarding myelin damage dissemination in time (DIT), MRI evidence is:
A new T2 and/or gadolinium-enhancing damage on follow-up MRI, compared to a baseline scan (irrespective of time since baseline). The 2005 revision had required at least 30 days to pass between the initial and first attack.
Simultaneous presence of asymptomatic gadolinium-enhancing and non-enhancing damage at any time.

Progressive primary MS (PPMS) has special diagnostic needs. Specifically, the revised McDonald Criteria call for at least one year of demonstrated progression (done prospectively or retrospectively), plus two of the following three findings:
Evidence of DIS in the brain, seen in at least one T2 lesion in the three key brain regions (periventricular, juxtacortical or infratentorial).
Evidence of DIS in the spinal cord, based on at least two T2 lesions DIS (>2 T2 damage).
Positive CSF involvement as seen in the presence of oligoclonal bands and/or a high IgG index.

### Gd+ Lesion

Gadolinium (“contrast”) is a chemical compound that is injected into a person's vein during an MRI scan. Gadolinium normally cannot pass from the bloodstream into the brain or spinal cord due to the blood-brain barrier. But during active inflammation within the brain or spinal cord, as during an MS relapse, the blood-brain barrier is disrupted, allowing gadolinium to pass through. Gadolinium can then enter the brain or spinal cord and leak into an MS lesion, lighting it up and creating a highlighted spot on an MRI. Such an MS lesion is called gadolinium-enhanced lesion or Gd+ lesion.

### T1 and T2 Lesions

T1 and T2 relate to different MRI methods used to generate magnetic resonance images. Specifically, T1 and T2 refers to the time taken between magnetic pulses and recording of an image. These different methods are used to detect different structures or chemicals in the central nervous system. T1 and T2 lesions refer to whether the lesions were detected using either the T1 or T2 method. A T1 MRI image supplies information about current disease activity by highlighting areas of active inflammation. A T2 MRI image provides information about disease burden or lesion load (the total amount of lesion area, both old and new).

### EDSS

The Expanded Disability Status Scale (EDSS) is a method of quantifying disability in multiple sclerosis and monitoring changes in the level of disability over time.

The EDSS scale ranges from 0 to 10 in 0.5-unit increments that represent higher levels of disability. Scoring is based on an examination by a neurologist.

EDSS steps 1.0 to 4.5 refer to people with MS who are able to walk without any aid and is based on measures of impairment in eight functional systems (FS):
pyramidal—muscle weakness or difficulty moving limbs
cerebellar—ataxia, loss of balance, coordination or tremor
brainstem—problems with speech, swallowing and nystagmus
sensory—numbness or loss of sensations
bowel and bladder function
visual function—problems with sight
cerebral functions—problems with thinking and memory
other.

A functional system (FS) represents a network of neurons in the brain with responsibility for particular tasks. Each FS is scored on a scale of 0 (no disability) to 5 or 6 (more severe disability). Reference is made to Kurtzke J F. Rating Neurologic Impairment in Multiple Sclerosis: An Expanded Disability Status Scale (EDSS). Neurology: 1983, Nov; 33(11):1444-52.

Multiple Sclerosis Impact Scale (MSIS-29)

The MSIS-29 version 2 is a 29-item, self-administered questionnaire that includes 2 domains: physical and psychological. Responses were captured on a 4-point ordinal scale ranging from 1 (not at all) to 4 (extremely), with higher scores reflecting greater impact on day-to-day life. The MSIS-29 takes about 5 minutes to complete and the questions are designed to determine the patient's views about the impact of MS on their day-to-day life during the past 2 weeks. Reference is made to Hobart J and Cano S (2009), “Improving the evaluation of therapeutic interventions in multiple sclerosis: the role of new psychometric methods”, Health Technol Assess; 13(12):iii, ix-x, 1-177. NS RO, to Hobart J, Lamping D, Fitzpatrick R, et al (2001), “The Multiple Sclerosis Impact Scale (MSIS-29): a new patient-based outcome measure”, Brain; 124(Pt 5):962-73.

SDMT or symbol digit Modalities is a sensitive and specific test to assess processing speed which is typically affected in cognitively impaired MS participants (Benedict et al. 2017, Mult Scler; 23(5):721-733). The test scoring is calculated on the number of correct answers in 90 seconds (maximum is 110 and minimum zero). Higher scores indicates improvement and lower score indicates worsening.

T25 FW or time 25-foot walk is the time to 6-month confirmed worsening by at least 20% in the timed 25-foot walk test. T25 FW is an objective quantitative test of neurological function (Fischer et al 1999, Mult Scler; 5:244-50) and is widely used in clinical MS trials to assess ambulation. It is an ambulation measurement assessing speed of walking: a time (in seconds) walk of 25 feet (7.62 meters).

Longer time indicates poorer upper limb function. 20% improvement is defined as 20% shorter time in seconds. 9HPT or the Nine Hole Peg Test (9HPT) is an objective quantitative test of neurological function (Fischer et al. 1999 Mult Scler; 5:244-50) and is widely used in clinical MS trials to assess dexterity. It is measured to assess both right and left arm scores, the metric is the time, in seconds, required to insert and remove 9 pegs.

Nfl or neurofilament light chain NfL is a component of the neuronal cytoskeleton and is released into the cerebrospinal fluid and subsequently into blood following neuro-axonal damage. It has been identified as a biomarker of disease activity (Kuhle et al 2019, Ann Clin Transl Neurol; 6(9):1757-1770), disease monitoring (Akgun et al 2019, Neurol Neuroimmunol Neuroinflamm; 6(3):e555), treatment response (Hauser et al 2020, N Engl J Med; 546-557) and to predict disease activity and disability worsening in participants with MS (Barro et al 2018, Brain 141:2382-2391, Kuhle et al 2019, Kapoor et al 2020 Neurology; 95(10):436-444, Jakimovski et al 2019 Ann Clin Transl Neurol; 6(9):1757-1770).

GAD-7 or general anxiety disorder-7 is a 7-item, self-rated scale (developed by Spitzer et al 2006, Arch Intern Med; 166:1092-7) which is used as a screening tool and severity indicator for GAD. Response options for the scale consist of a 4-point Likert Scale: 0: not at all; 1: several days; 2: more than half the days; 3: nearly every day. It has a global score ranging from 0-21. Higher score means higher severity of anxiety symptoms.

PHQ-9 or patient health questionaire-9 is a 9-item reliable and valid depression module from the full PHQ. This self-administered tool is used for screening, diagnosing, monitoring and measuring the severity of depression. Additionally, it is used to making criteria-based diagnoses of depressive disorders (Kroenke et al 2001, J Gen Intern Med; 16:606-13).

The PHQ-9 scores can range from 0 to 27, since each of the 9 items can be scored from 0 (not at all) to 3 (nearly every day). PHQ-9 scores of 5, 10, 15, and 20 represented mild, moderate, moderately severe, and severe depression, respectively.

BPI-SF or brief pain inventory-short form is a 15-item shorter version of Brief Pain Inventory (Cleeland and Ryan 1994, Ann Acad Med; 23(2):129-59). It is used to assess the severity of pain and the impact of pain on daily functions. Along with severity of pain, location of pain, pain medications and amount of pain relief in the past 24 hours, BPI-SF also includes seven-item interference scale to assess the extent to which pain interferes with general activity, mood, walking, work, relationship with others, sleep, and enjoyment of life. It has a 10-point response option, ranging from 0 (does not interfere) to 10 (completely interferes). Global score ranges from 0 to 10, where lower scores represent lower pain.

FSIQ-RMS™ or the fatigue syndromes and impacts questionnaire—relapsing multiple sclerosis measures fatigue symptoms and impacts in RMS. This reliable and valid tool comprises of 20 items organized with two symptom domains (energy, muscle weakness) and seven impact domains (daily activities, cognition, emotions, physical impact, self-care, sleep and social impact). Recall period of 24 hours to capture symptoms and 7 days to capture impact. The FSIQ-RMS scores by domains and subdomains. Global score ranges from 0 to 100 where higher score represents greater fatigue (Hudgens et al 2019, Value Health; 22:453-466).

HUI-III™ or health utilities index®) is a family of generic health profiles and preference based systems, for the purpose of measuring health status, reporting health-related quality of life, and producing utility scores (Feeny et al 2002, Med Care; 40(2):113-28). The HUI-III measures eight HRQoL domain areas including vision, hearing, speech, ambulation/mobility, pain, dexterity, emotion, and cognition. This study will implement the self-reported version and participants will respond to their “usual health” in terms of the recall period. Answers to HUI questionnaires enable mapping responses to specific levels. HUI-III has following domains (levels for each domain):
Vision (6 levels)
Hearing (6 levels)
Speech (5 levels)
Ambulation (6 levels)
Dexterity (6 levels)
Emotion (5 levels)
Cognition (6 levels)
Pain (5 levels)

A decrease in HUI score means that the conditions have worsened.

### Disease-Modifying Therapy (DMT)

The term “disease-modifying therapy” is used because there is still no curative treatment for multiple sclerosis (MS), but several disease-modifying drugs (DMDs) have been approved for MS. Generally, DMTs for RMS decrease the frequency and/or seriousness of relapses. Thus, DMTs are not a cure for RMS patients, but they can reduce how many relapses someone has and how serious they are. DMTs include, without limitation treatment with DMDs such as interferon beta, glatiramer acetate, teriflunomide, mitoxantrone, dimethyl fumarate, cladribine, fingolimod, siponimod, ponesimod, alemtuzumab, daclizumab, natalizumab, ofatumumab, ocrelizumab and rituximab.

According to the present invention a DMT has a “lack of efficacy” if it is not stopping or not appropriately slowing down disease progression. This is the case, for example if a patient who is on a DMT shows signs of disease activity, such as relapses or lesions.

According to the present invention, a “lack of tolerability” of a DMT relates to the presence of adverse events such as headache, dizziness, nausea, infections (such herpes zoster), macular edema, infusion-related reactions or recurrent infections.

### B Cell-Depleting Therapy

The term “B cell-depleting therapy” as used herein refers to any therapy resulting in B cell depletion, including CD19-/CD20-depleting therapies such as B cell-depleting therapies based on anti-CD20 mAbs. In particular, in B cell-depleting therapies based on anti-CD20 mAbs depletion of B cells is achieved by administration of monoclonal antibodies that target CD20-expressing B cells, e.g. alemtuzumab, daclizumab, natalizumab, ofatumumab, ocrelizumab and rituximab.

### Loading Dose

A loading dose is an initial dose of a drug, preferably an initial higher dose, that may be given at the beginning of a course of treatment (e.g. a DMT) before succeeding with a maintenance dose, preferably dropping down to a lower maintenance dose.

### Neurologically Stable

A clinical state characterized by lack of change in mental status or level of consciousness. This state may comprise control of seizures; absence of new neurologic defects, e.g. aphasia, ataxia, dysarthria, paresis, paralysis, visual field loss, or blindness, and is defined as neurologically stability.

### Rebound

Severe disease reactivation after the withdrawal of DMT that exceeds a patient's pre-DMT baseline is considered a rebound event. Reference is made to Barry et al., Fingolimod Rebound: A Review of the Clinical Experience and Management Considerations. Neurol Ther (2019) 8:241-250.

### Breakthrough Disease

For the purpose of this invention, breakthrough disease is defined as:
at least one documented relapse during the previous year or two relapses during the previous two years,
presence of at least one Gd+ lesion on an MRI scan within the last 12 months, and/or
presence of new or enlarging T2 lesions within the last 12 months under a DMT.

“B cell inhibitor” as used herein generally may relate to any substance that abolishes, reduces or attenuates biological B cell functions. The B cell inhibitor may interrupt signal transduction pathways that are necessary for biological B cell functions, e.g. cytokine secretion or responses to cis and/or trans stimulation. The B cell inhibitor may also interfere with the generation of B cells from stem/progenitor cells or negatively affect their maturation. Moreover, the B cell inhibitor may act by inhibiting the cross-talk with other cell populations such as T cells. Alternatively, the B cell inhibitor may deplete B cells by sequestration (e.g. into lymphoid tissues such as the spleen) or by lysis, e.g. through CDC, ADCC, phagocytosis or other processes. Several subsets of B cells may express CD20.

B cell as used herein may relate to a type of white blood cell of the lymphocyte subtype. B cells function in the humoral immunity component of the adaptive immune system by secreting antibodies such as immunoglobulins (e.g. IgG). Additionally, B cells may present antigens and secrete cytokines. B cells, unlike T cells and natural killer cells, express B cell receptors (BCRs) on their cell membrane. BCRs allow the B cell to bind to a specific antigen against which it will initiate an antibody response.

T cell inhibitor as used herein may relate to any substance that abolishes, reduces and/or attenuates biological T cell functions. The T cell inhibitor may interrupt signal transduction pathways that are necessary for biological T cell functions, e.g. cytokine secretion or responses to cis and/or trans stimulation. The T cell inhibitor may also interfere with the generation of T cells from stem/progenitor cells or negatively affect their maturation. Moreover, the T cell inhibitor may act by inhibiting the cross talk with other cell populations such as B cells. Alternatively, the T cell inhibitor may deplete T cells by sequestration (e.g. into lymphoid tissues such as the spleen) or by lysis, e.g. through CDC, ADCC, phagocytosis or other processes.

T cell as used herein may relate to a type of lymphocyte which develops in the thymus gland. T cells can be distinguished from other lymphocytes by the presence of a T cell receptor on the cell surface.

### Examples

### Example 1

Bruton's Tyrosine Kinase (BTK) Inhibitor LOU064 in HumanMOG-Induced Experimental Autoimmune Encephalomyelitis

There is no known naturally occurring MS-like disease in rodents or non-human primates. Following immunization of susceptible rodent strains with CNS-specific myelin proteins emulsified in complete Freund's adjuvant (CFA) an induced disease model develops. Experimental autoimmune encephalitis (EAE) mimics many of the pathological phenotypes of MS with focal, demyelinating lesions of the CNS white matter. In the C57BL/6 mouse there are two variants of myelin oligodendrocyte glycoprotein (MOG)-induced EAE depending on whether the human (HumanMOG) or rat (RatMOG) recombinant protein sequences are used (Lyons J.A. et al. (1999) European Journal of Immunology 29:3432-3439; Oliver A. R. et al. (2003) J Immunology 171: 462-468). Minor amino acid residue differences result in HumanMOG-induced EAE being exquisitely B-cell dependent as the APC, whilst RatMOG-induced EAE is B-cell independent and requires dendritic cells as the dominant APC population (Lyons et al 1999; Molnarfi N. et al. (2013) Journal of Experimental Medicine 210(13):2921-2937).

In HumanMOG-induced EAE the BTK inhibitor LOU064 was dosed twice daily (b.i.d.) at 3 mg/kg or 30 mg/kg at 12 hr intervals. Prophylactic dosing of LOU064 was initiated 6 hours prior to MOG/CFA immunization and continued until study end.

EAE was assessed using a scoring system, outlined in Table 1. Clinical scores and body weights were assessed daily throughout the experiment. Prior to treatment start animals were randomized so that all groups were comparable for clinical profile and body weights.

TABLE 1
EAE scoring criteria

| Score | Observation |
|---|---|
| 0 | Normal appearance/no detectable signs of disease |
| 0.5 | Distal limp tail |
| 1 | Complete tail paralysis |
| 1.5 | Hind limb weakness - grid test positive |
| 2 | Unilateral partial hind limb paralysis - impaired righting reflex |
| 2.5 | Bilateral partial hind limb paralysis |
| 3 | Complete bilateral hind limb paralysis |
| 3.5 | Fore limb weakness and complete bilateral hind limb paralysis |
| 4 | Quadriplegia/Moribund |
| 5 | Death from EAE |

According to the animal regulations, humane endpoints for EAE mice were score 3 (>7 days), score 3.5 (>3 days), or immediately if score 4 was reached.

LOU064 (30 mg/kg p.o. b.i.d.) inhibited inflammation-induced cachexia and significantly reduced clinical symptoms of EAE (FIG. 1). The compound was well tolerated in all animals.

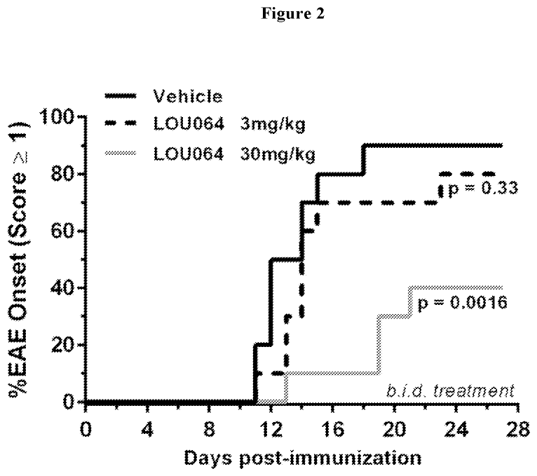

Additional analysis revealed that LOU064 efficacy was associated with a reduction in the frequency of EAE onset with many animals completely protected from disease (FIG. 2).

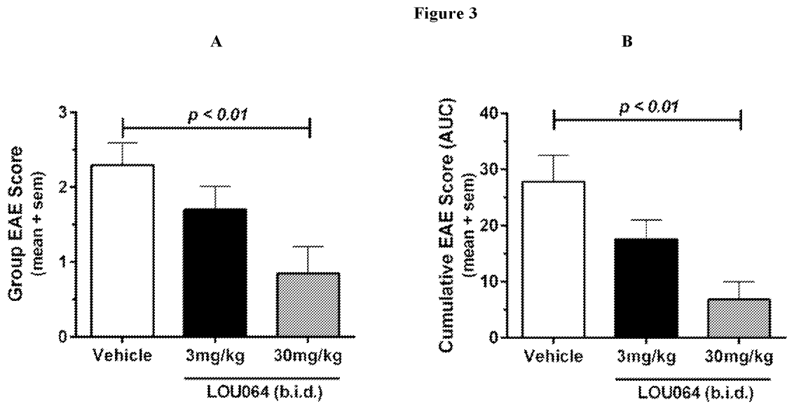

BTK inhibition also reduced group EAE score (peak neurological paralysis) and total disease burden during the entire experimental period (FIG. 3). The concentrations of LOU064 present in blood 1, 5 and 8 hours after compound b.i.d. dosing are shown in Table 2. The exposure in blood shows the expected levels at the 1 hour timepoint with a fast decrease over the 5 and 8 hour timepoints, as well as a dose-proportional increase from 3 to 30 mg/kg b.i.d. dosing. The compound levels in total brain homogenate are very low and mainly detectable at the early timepoint. Similarly, the levels in cerebrospinal fluid (CSF) are low.

TABLE 2
Levels of LOU064 in mouse tissues

| | 3 mg/kg | | | 30 mg/kg | | |
|---|---|---|---|---|---|---|
| | 1 h | 5 h | 8 h | 1 h | 5 h | 8 h |
| Blood (nM) | 28.2 ± 11.7 | 0.4 ± 0.04 | 0.2 ± 0.07 | 371.2 ± 149 | 19.4 ± 9.9 | 3.4 ± 2.3 |
| Brain (pmol/g) | 2.3 ± 2.2 | <0.5 | <0.5 | 13.5 ± 6.0 | 0.7 ± 0.02 | <0.5 |
| CSF (nM) | 3.0 ± 3.3 | 0.5^{a} | <0.5 | 8.4 ± 3.1 | 0.5^{a} | <0.5 |

Blood, brain and CSF levels of LOU064 after oral dosing of 3 and 30 mg/kg b.i.d. The LLOQ were 0.2 nM in blood, 0.5 μmol/g in brain homogenate and 0.5 nM in CSF. Shown are averages ±SD from 4 animals for the 1 h timepoints and from 3 animals for the 5 and 8 h timepoints. Where denoted with ^{a}, the values were derived from one animal of three that was above the LLOQ.

BTK occupancy in spleen was determined at 1, 5 and 8 hours after b.i.d. oral dosing of LOU064 (FIG. 4). BTK occupancy in spleen was maximal for both doses and showed a decay after a 3 mg/kg dose with a more sustained occupancy after the 30 mg/kg dose. These levels of BTK occupancy were comparable to other studies performed in mice.

The BTK occupancy in inguinal lymph nodes is shown in FIG. 5. BTK occupancy was maximal for both doses and showed a decay after a 3 mg/kg dose with a more sustained occupancy after the 30 mg/kg dose. These levels of BTK occupancy were comparable to other studies performed in mice with LOU064.

BTK occupancy was assessed in brain homogenates that had been prepared for compound exposure analysis (FIG. 6). The dose group receiving 30 mg/kg b.i.d. LOU064 showed maximal BTK occupancy with a decline over the dosing interval. The 3 mg/kg dose led to only minimal BTK occupancy at the 1 hour timepoint. Variability in brain BTK occupancy might be due to the fact that the analysis was performed on the remainder of the homogenates prepared for compound level assessment.

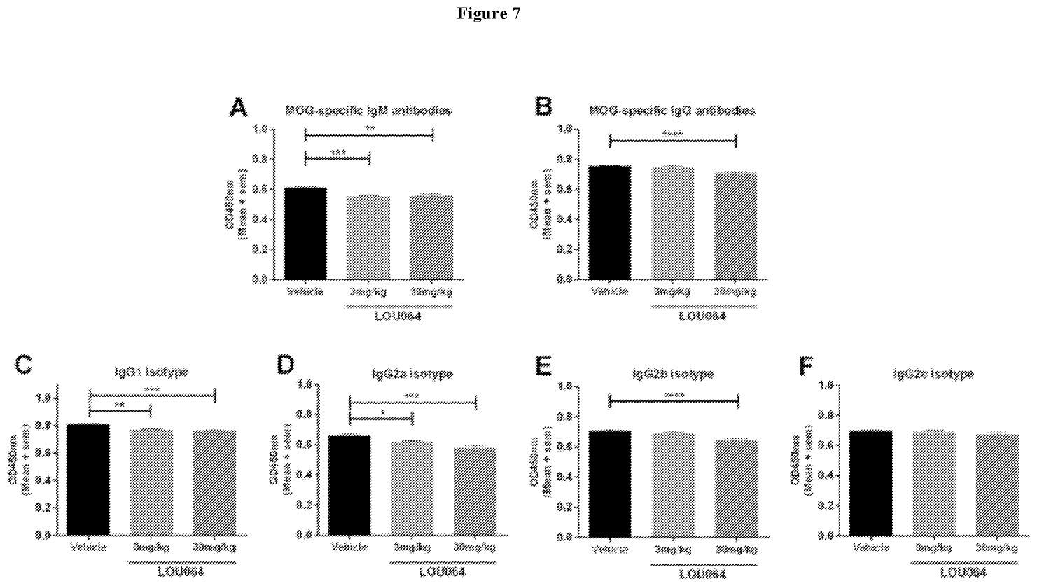

Ex vivo analysis of MOG-specific antibody responses in serum revealed only a small but statistically significant reduction in IgM and IgG levels (FIG. 7). In the HumanMOG-induced EAE model the (auto)antibody response is weakly pathogenic. The discrepancy between EAE score efficacy and only minor modulation of antibodies suggests that B-cell antigen presentation is the key driver for the neuroinflammatory process.

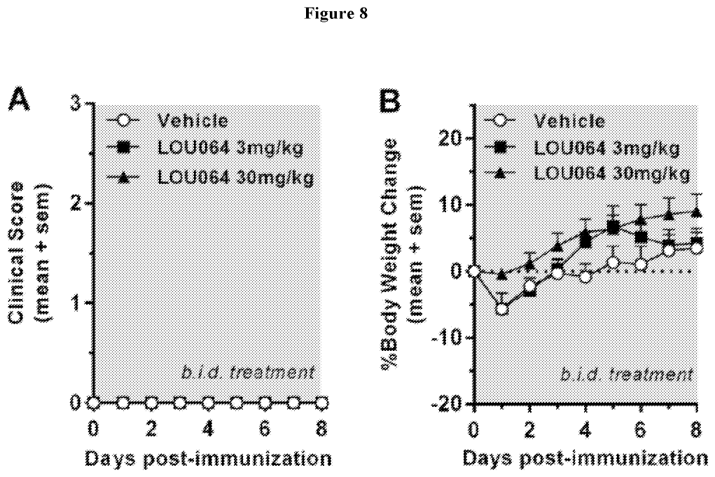

Using a short eight-day (pre-disease onset) EAE protocol, the immune priming mechanisms during the HumanMOG model were investigated. Prophylactic treatment with LOU064 was associated with improved weight gain suggesting that inflammation-induced cachexia in the model was modulated (FIG. 8). As previously described, no adverse events were observed with either dose group.

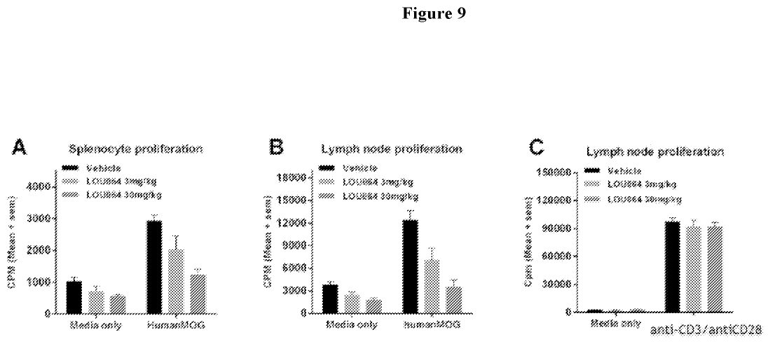

Ex vivo MOG-induced recall proliferation responses were studied using isolated splenocytes and draining lymph node cells collected on day 8 post-immunization. In vivo treatment resulted in a dose-dependent reduction of proliferation in both immune cell compartments (FIG. 9). In contrast, plate-bound anti-CD3/CD28 polyclonal stimulation of lymph node cells was unaffected by the in vivo treatment. This data suggests a specific inhibition of B-cell antigen presentation function rather than broad immunosuppression.

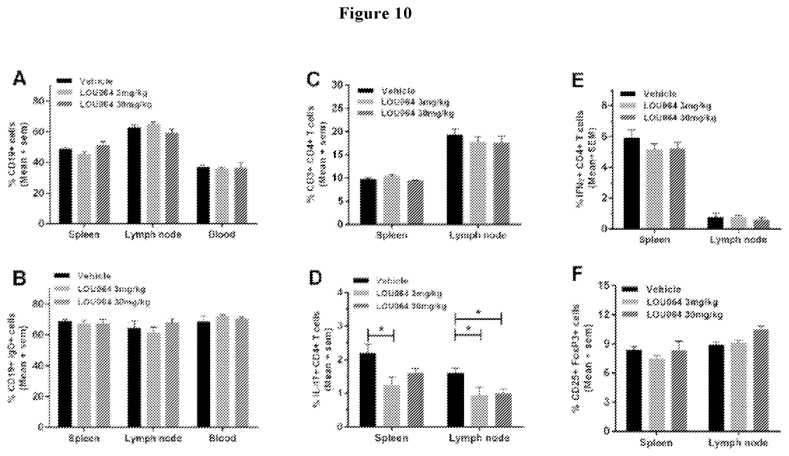

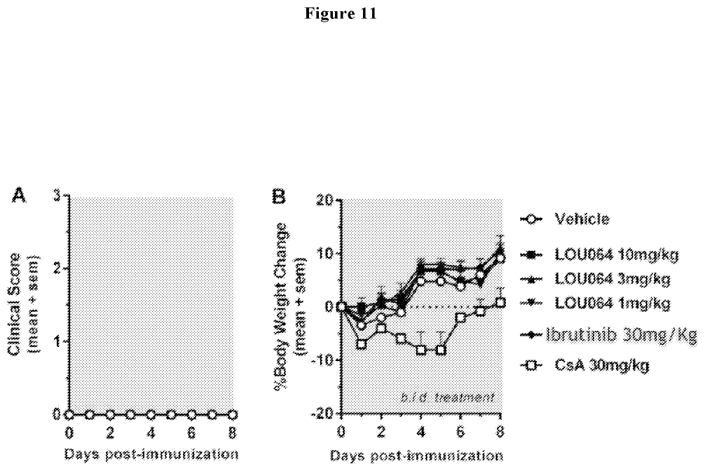

Ex vivo analysis of isolated splenocytes, lymph node cells and blood revealed no significant changes in total B-cell populations (FIG. 10). Analysis of CD4+ T-cells revealed that only the Th17 population was reduced following LOU064 treatment whilst Th1 and regulatory T-cells were unchanged.

To elucidate further whether BTK inhibitor efficacy was directly linked to reduction in B-cell antigen-presenting function compounds were tested in recombinant RatMOG-induced EAE model. This EAE model shares many of the characteristics of recombinant HumanMOG-induced EAE with MOG-specific T-cells infiltrating the CNS and resulting in neurological paralysis. However, RatMOG-induced EAE is B-cell independent and the dendritic cells are the dominant antigen-presenting cells type. Therefore, BTK inhibitors would be predicted to have no significant efficacy in RatMOG-induced EAE unless drug treatment was associated with a broader, non-specific, immune suppression. In addition to LOU064, which was used at 1 mg/kg, 3 mg/kg and 10 mg/kg, the reference compound ibrutinib was tested.

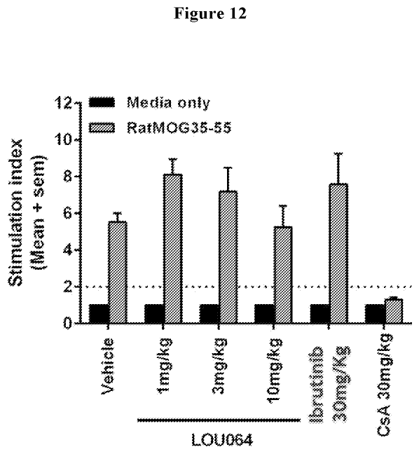

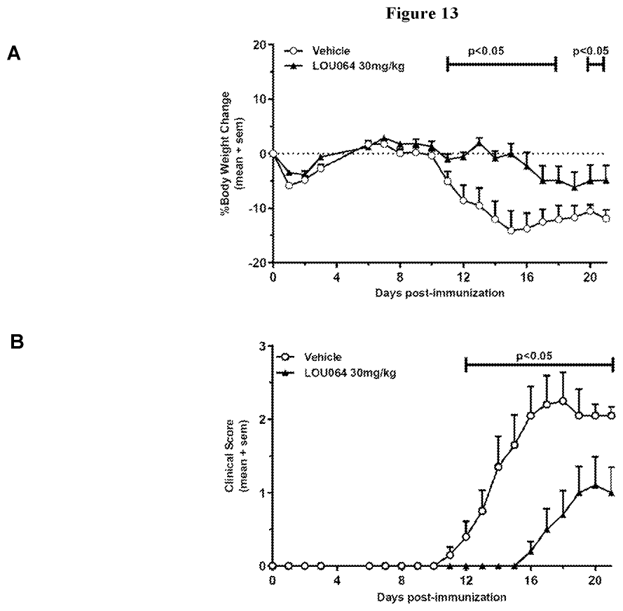

Cyclosporin A (CsA) acted as a positive control for direct T-cell immunosuppression. Using the short eight-day (pre-disease) EAE protocol the immune priming mechanisms during RatMOG model were investigated, no adverse events were observed with either BTK inhibitor. Ex vivo MOG-induced recall proliferation responses were studied using isolated splenocytes collected on day 8 post-immunization. In vivo BTK inhibitor treatment had no effect on recall responses (FIG. 12). In contrast, CsA profoundly inhibited T-cell recall proliferation. This data suggests BTK inhibition mediated immune modulation is highly selective and directly relevant to B-cells acting as antigen-presenting cells in the (auto)immune priming phase.

### Conclusion and Discussion

These studies have utilized two distinct EAE models in the C57BL/6 mouse. The recombinant HumanMOG-induced EAE model is B-cell dependent and sensitive to anti-CD20 B-cell depletion, whereas the RatMOG-induced EAE model is B-cell independent with dendritic cells acting as the key antigen presenting cell.

It was demonstrated that the low molecular weight BTK irreversible (covalent) inhibitor LOU064 unexpectedly was highly effective at inhibiting HumanMOG-induced EAE induction. Efficacy on clinical score and inflammation-induced cachexia was closely linked to sustained high levels of BTK occupancy in tissues. Mechanistically, the experimental findings are consistent with suppression of B cell-dependent (auto)antigen presentation during the immune priming phase resulting in lower frequencies of pathogenic Th17 cells. The disconnect between significant clinical score efficacy and minor reductions of (auto)antibodies suggests this is not a critical pathogenic mechanism.

The evidence from the RatMOG-induced EAE model showed BTK inhibition did not impact (auto)immune T-cell responses in the immune priming phase. Therefore it is possible to conclude that BTK inhibitor (LOU064) efficacy is not the result of broad (non-selective) immune suppression.

In conclusion, surprisingly the covalent BTK inhibitor LOU064 demonstrated a highly selective mechanism of action, unexpectedly resulting in excellent and superior efficacy on a pathogenic process known to be highly relevant for treating multiple sclerosis in human.

### Example 2

### Bruton's Tyrosine Kinase (BTK) Inhibitor LOU064 in RatMOG-Induced Experimental Autoimmune Encephalomyelitis

It has been reported that pharmacological BTK inhibition or genetic BTK deficiency ameliorates preclinical mouse EAE suggesting that a BTK inhibitor may achieve efficacy due to its effects on B cells, as well as on myeloid cells (Torke S. & Weber M.S. (2020) Expert Opinion on Investigational Drugs 29:1143-1150; Mangla A. et al. (2004) Blood 104(4):1191-7 and Example 1). While the previous study with LOU064 focused on an experimental autoimmune encephalomyelitis (EAE) model that is B cell driven (Example 1), the present study further extends this evidence by showing that LOU064 shows efficacy in the B cell-independent RatMOG-EAE, likely by its action on myeloid cells.

LOU064 Treatment Reduced EAE Development In RatMOG-induced EAE the BTK inhibitor LOU064 was dosed twice daily (b.i.d.) at 30 mg/kg at 8/16 hour intervals. Dosing of LOU064 was initiated 4 hours prior to MOG/CFA immunization and continued twice daily until study end. LOU064 inhibited inflammation-induced clinical symptoms of EAE and cachexia (FIG. 13). The compound was well tolerated in all mice.

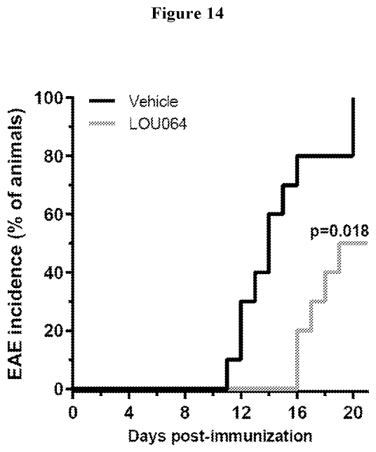

Additional analysis revealed that LOU064 efficacy was associated with a reduction in the incidence of EAE and a delayed onset with many mice completely protected from disease (FIG. 14) up to study termination (p value=0.018).

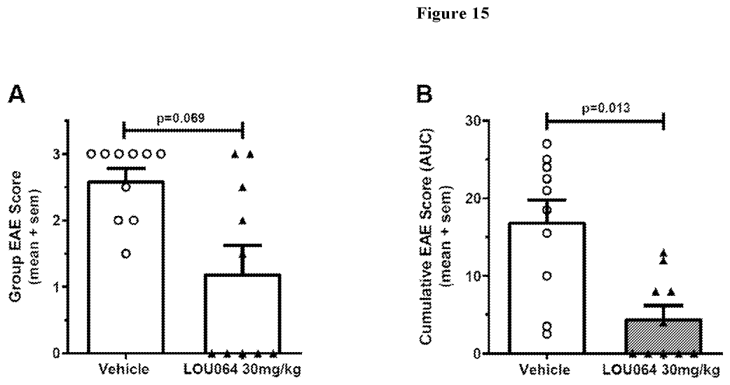

BTK inhibition also significantly reduced group EAE scores (peak neurological paralysis p value=0.069) and total disease burden (p value=0.013) during the entire experimental period (FIG. 15).

### LOU064 Treatment Reached its Pharmacological Target

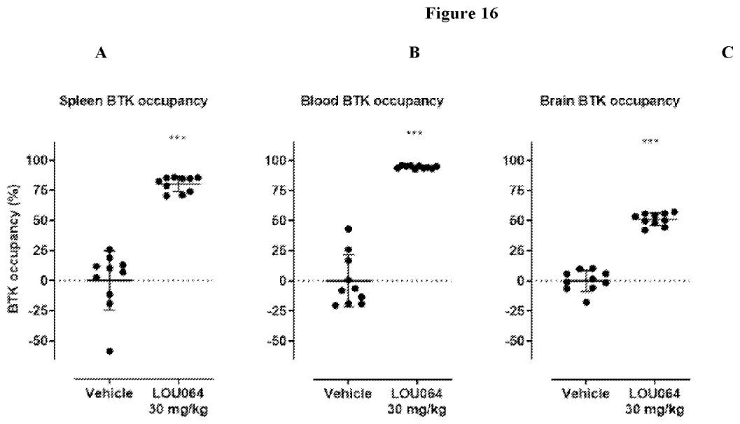

FIG. 16 shows terminal BTK occupancy in spleen, blood and brain sampled from mice on the day of termination, 16 h after the last dose. The trough BTK occupancy levels in spleen and blood were in a range indicative of maximal BTK occupancy at the time of peak LOU064 exposures. It was shown to be influenced by re-synthesis of free BTK once systemic exposure of LOU064 has waned (Angst et al. 2020). These levels of BTK occupancy were comparable to previous studies performed in mice.

BTK occupancy assessed in brain homogenates showed intermediate levels, suggesting that significant (p values <0.001 for spleen, blood and brain) but possibly submaximal brain BTK occupancy was reached at peak blood exposure.

### LOU064 Treatment had No Impact on Autoantibody Levels

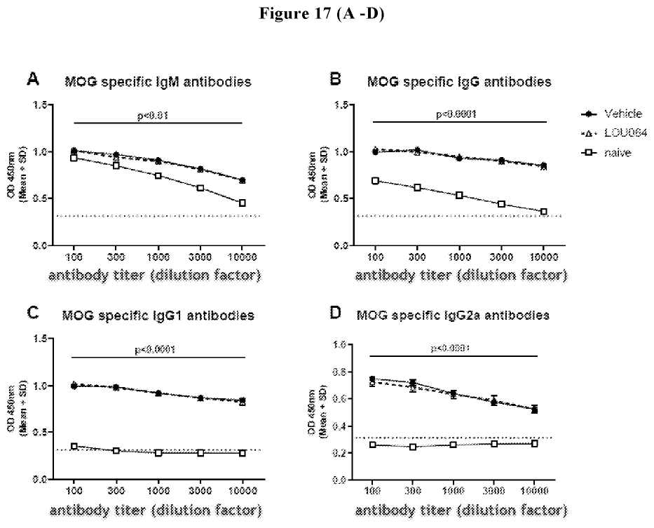

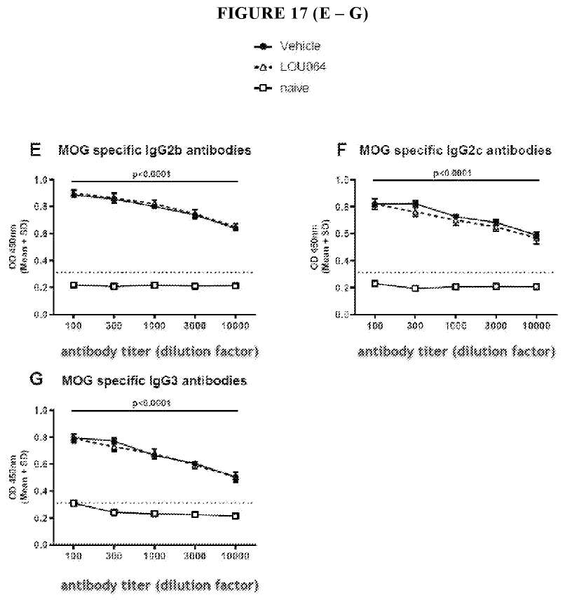

FIG. 17 shows the levels of MOG-specific autoantibodies in serum 16 hours after the last dosing on the day of termination (day 21). In immunized mice, treated with either vehicle or LOU064, the MOG-specific autoantibody levels, total IgM and IgG subclasses were markedly higher as compared to naive mice. LOU064 treatment had no effect on the MOG-specific IgM and IgG responses in serum compared to vehicle treated mice. These results are in line with those observed in the human MOG EAE model, as described in Example 1 above.

### LOU064 Treatment Tended to Reduce Serum NF-L Levels

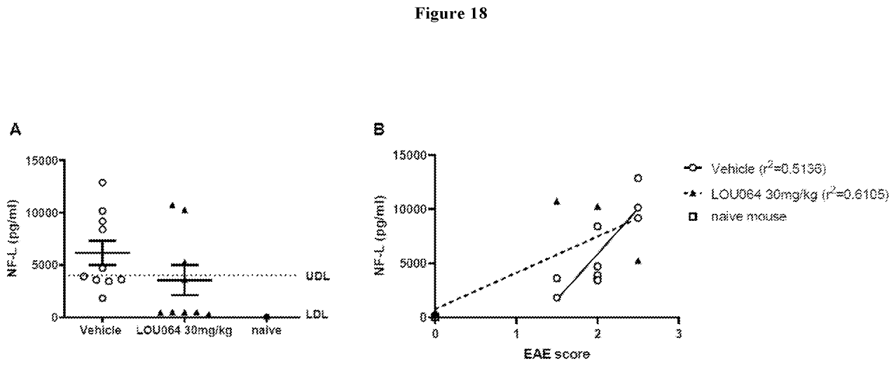

FIG. 18 shows the levels of NF-L in serum. In immunized mice, vehicle or LOU064-treated, the mean NF-L level was markedly higher as compared to naive mice. Moreover, the vehicle treated-group showed a significant correlation between the increase in clinical score and the serum NF-L levels (p value=0.0006). This was observed in the LOU064-treated group with a non-significant trend towards a lower mean NF-L level as compared to the vehicle-treated group.

### Evidence of BTK Expression in Tissues Via IHC

FIG. 19 shows representative examples of IHC stainings for the expression of BTK in lymph nodes (positive control; A and B) and in the brain of naive mice (C and D). In these mice, BTK is expressed in B cell follicles in the lymph nodes (A), in particular in some disseminated cells throughout the paracortex of lymph nodes (B). In naive mouse brain, in particular in the corpus callosum, BTK could not be detected (C and D).

### Conclusion and Discussion

The main objective of the present study was to assess the efficacy of the potent and selective BTK covalent inhibitor (LOU064) in a B-cell independent EAE model. In this study, LOU064 was given at the dose of 30 mg/kg b.i.d. which demonstrated full BTK occupancy in blood and spleen but only partial BTK occupancy in the brain.

The results obtained show that LOU064 was indeed efficacious at reducing the severity of RatMOG-induced EAE, without impacting the parallel IgM and IgG responses.

Altogether, these results show a therapeutic efficacy for LOU064 independently from its ability to inhibit BTK in B cells. As myeloid cells such as macrophages, neutrophils or mast cells are also known to express BTK (Torke et al., 2020) a key pathogenic role can be suspected for these cells in the model used. This is in line with recent preclinical and clinical observations clearly indicating beneficial effects in targeting peripheral myeloid cells for the control of CNS inflammation (Ifergan I. and Miller S.D. (2020) Front Immunology 11:571897).

While a driver for the efficacy of LOU064 in the present study, using the RatMOG EAE model, might be the inhibition of peripheral BTK expressing immune cells of the myeloid type, such as macrophages, it has been unexpectedly shown that the level of BTK occupancy detected in the brain could also contribute to the overall treatment efficacy. Since the histological analysis did not reveal BTK expression in the brains of healthy mice, it can be concluded that the level of BTK occupancy measured in the brains of EAE mice reflects EAE-induced CNS infiltration by BTK-expressing cells.

The fact that the present study shows no impact for LOU064 on the EAE-induced IgM/IgG responses is consistent with previous observations on the level of the anti-MOG antibody response (Example 1). In conclusion, the covalent BTK inhibitor LOU064 unexpectedly showed excellent efficacy on a pathogenic process known to be highly relevant for treating multiple sclerosis in humans. Especially advantageous results have been surprisingly achieved with an EAE dosage of 30 mg/kg b.i.d. converting into a human dose of 100 mg b.i.d.

### Example 3: LOU064 Brain Permeability

Passive permeability measurement using canine P-gp knockout MDCK cell monolayers (MDCK-LE V2)

A cell-based assay was used to assess the passive permeability of drug candidates in the context of gastro-intestinal absorption. A MDCK cell line where the endogenous canine Mdr1 (cMdr1) gene encoding P-gp has been knocked out is grown to form monolayers on a 96-well Transwell plate. Compounds are loaded in cassettes of three at a concentration of 10 μM each to the apical compartment and following a period of two hours incubation the amount of compound appearing in the basal chamber is quantitated by tandem mass spectrometry.

In particular, a MDCK cell line was generated by knocking-out the endogenous canine Mdr1 (cMdr1) gene. A subclone of this cell line was used to relate passive permeability to the fraction absorbed in human intestine using a set of 37 commercial compounds for which the fraction absorbed was known. The assay is able to estimate the fraction absorbed in human based on the Papp measured in the assay.

### Methods

Samples were subject to RapidFire/MS/MS analysis and LC/MS/MS analysis. Rapidfire and LC/MS/MS conditions are noted below.
Mass spectrometer: Sciex QTRAP5500
Autosampler: Shimadzu SIL-30ACmp
HPLC pump: Shimadzu LC-30AD
Column: Phenomenex Kinetex Polar C18 2.1×30 mm, 2.6 μm
Oven temperature: 50° C.
Injection volume: 2 μL injection
Mobile Phase A: water containing 0.1% (v/v) formic acid
Mobile Phase B: acetonitrile containing 0.1% (v/v) formic acid and 4% (v/v) water

| Gradient: | | | |
|---|---|---|---|
| time | % A | % B | Flow(μL\min) |
| 0 | 98 | 2 | 800 |
| 0.2 | 98 | 2 | 800 |
| 1.0 | 40 | 60 | 800 |
| 1.3 | 0 | 100 | 800 |
| 1.7 | 0 | 100 | 800 |
| 1.71 | 98 | 2 | 800 |
| 1.95 | 98 | 2 | 800 |

Detector: SCIEX API5500 QTrap Parameters:
Source: ESI
IonSpray Voltage: 4500V (−4500V in negative mode)
Ion source Gas1: 60 psi
Ion source Gas2: 40 psi
Temperature: 450° C.
Curtain Gas: 30 psi
Collision Gas: 9 psi

All MS parameters such as parent mass, product mass, de-clustering potential (DP), collision energy (CE), and others are acquired using the auto-tuning application, DiscoveryQuant-Optimize. The scan time is 0.025 s.

A faster alternative to a classical HPLC frontend instrument is the RapidFire 360 system (Agilent Technologies) equipped with a C4 (RFCP4A) cartridge. The device is connected to a tandem mass spectrometer in a manner similar to an HPLC. The Rapid Fire performs a solid phase extraction with a minimal chromatography. The approach is much faster than traditional LC, but the LLOQ can increase and some compounds are poorly retained on the cartridge, therefore some compounds might need to be reprocessed using the classical LC approach.
RapidFire parameters:
RapidFire: Agilent RF360 parameters:
RapidFire cycle duration:
Aspirate: 500 ms (˜20 μL)
Load/Wash: 4000 ms, with a flow rate of 1.5 mL/min using 0.1% (v/v) formic acid in water
Elute: 3000 ms with a flow rate of 1.0 mL/min using 0.1% (v/v) formic acid in acetonitrile
Re-equilibrate: 500 ms, with a flow rate of 1.25 mL/min using 0.1% (v/v) formic acid in water
RapidFire cartridge column type: C4 (RFCP4A)

### Results

For LOU064 a high permeability of Papp=37.3 was found with high recovery in a MDCK trans-well assay.

### Example 4

### Safety of LOU064

The safety of LOU064 has been tested in Phase I and Phase II pharmacokinetic and clinical pharmacology healthy subject studies and in Phase II/Phase III clinical studies conducted with patients suffering from indications other than MS, particularly chronic spontaneous urticaria (CSU) and Sjoegren's Syndrome (SjS).

### Short-Term Safety of LOU064 in Phase I Clinical Study

Short-term safety of LOU064 as a single dose or as multiple doses for up to 18 days covering the dose range from 0.5 mg to 600 mg for up to 18 days and further at 100 and 200 mg b.i.d. for up to 12 days has been shown in Phase I clinical studies (Kaul, M. et al. (2021). Remibrutinib (LOU064): A selective potent oral BTK inhibitor with promising clinical safety and pharmacodynamics in a randomized phase I trial. Clinical and Translational Science. 10.1111/cts.13005).

### Summary of Safety and Tolerability in Phase 2b Study in CSU Subjects (Interim Results)

In a dose range finding study conducted in CSU subjects the following doses are being tested: 10, 35 and 100 mg q.d. as well as 10, 25 and 100 mg b.i.d. and placebo over 12 weeks.

LOU064was well-tolerated across the whole dose range, with most AEs being mild in severity and having no apparent dose-dependent pattern. Serious AEs and the most frequent AEs are listed in Table 3 by preferred term and primary system organ class. Overall, 58.1% of patients taking any dose of LOU064 had at least one AE: 38.6% of patients on LOU064 had mild AEs, 16.9% of patients had moderate AEs, and 2.6% of patients had severe AEs. In the placebo arm, 42.9% of patients reported at least one AE: 33.3% of patients on placebo had mild AEs, 9.5% had moderate AEs and 0.0% had severe AEs. There were no deaths during the study. Serious AEs were reported by five patients (1.9%) receiving any LOU064 dose as compared with no patients (0.0%) who received placebo: a renal abscess was reported by one patient which led to discontinuation of treatment (on Day 29, 25 mg b.i.d.); one patient reported worsening of lymphadenopathy (present before the start of the study but worsened on Day 12, 10 mg q.d.); ureterolithiasis was reported for one patient during the treatment-free follow-up period (on Day 87, 10 mg b.i.d.); and two patients experienced a flare/aggravation of their CSU (one on Day 30, leading to discontinuation, 10 mg b.i.d. and one on Day 5, 25 mg b.i.d.).

Infections and infestations were the most common AEs by primary system organ class with 24.0% of patients receiving any LOU064 dose versus 21.4% receiving placebo. Headache (9.7% in any LOU064 dose versus 14.3% placebo) and nasopharyngytis (8.6% versus 7.1%) were the most frequently reported AEs (occurring in ≥10% of the patients in any treatment arm).

Overall, 2.6% of patients (n=7) for any LOU064 dose had an AE leading to study drug discontinuation versus 0.0% for placebo (n=0). There were no significant laboratory findings, including blood cell counts in any of the trial groups during the study.

The analysis of laboratory data did not reveal significant findings. In total, there were three Common Terminology Criteria for AEs Grade 3 events (none higher), all asymptomatic and resolved without medical intervention. One patient had creatinine elevation at Week 8 after starting a ketogenic diet (10 mg q.d.); values normalized after stopping the diet. There was one neutrophil count decrease at Week 12 in a patient with a history of lymphocytopenia (35 mg q.d.); values improved during the treatment-free follow-up period. There was one alanine transaminase elevation at Week 8 (100 mg q.d.); values returned to normal while on treatment.

TABLE 3
Safety summary of treatment-emergent adverse events, serious adverse events and exposure

| | LOU064 | LOU064 | LOU064 | LOU064 | LOU064 | LOU064 | Any | |
|---|---|---|---|---|---|---|---|---|
| | 10 mg | 35 mg | 100 mg | 10 mg | 25 mg | 100 mg | LOU064 | |
| | q.d. | q.d. | q.d. | b.i.d. | b.i.d. | b.i.d. | dose | Placebo |
| | N = 44 | N = 44 | N = 47 | N = 44 | N = 43 | N = 45 | N = 267 | N = 42 |
| | n (%) | n (%) | n (%) | n (%) | n (%) | n (%) | n (%) | n (%) |
| Duration of | 38 | 38 | 39 | 37 | 38 | 31 | 221 | 34 |
| exposure ≥12 | (86.4) | (86.4) | (83.0) | (84.1) | (88.4) | (68.9) | (82.8) | (81.0) |
| weeks | | | | | | | | |
| Subject- | 10.1 | 10.1 | 10.9 | 9.8 | 9.8 | 9.3 | 59.9 | 9.5 |
| year | | | | | | | | |
| Patients | 1 | 0 | 0 | 2 | 2 | 0 | 5 | 0 |
| with | (2.3) | (0.0) | (0.0) | (4.5) | (4.7) | (0.0) | (1.9) | (0.0) |
| SAE(s) | | | | | | | | |
| Lymphadenopathy | 1 | 0 | 0 | 0 | 0 | 0 | 1 | 0 |
| (PT) | (2.3) | (0.0) | (0.0) | (0.0) | (0.0) | (0.0) | (0.4) | (0.0) |
| Renal | 0 | 0 | 0 | 0 | 1 | 0 | 1 | 0 |
| abscess | (0.0) | (0.0) | (0.0) | (0.0) | (2.3) | (0.0) | (0.4) | (0.0) |
| (PT) | | | | | | | | |
| Ureterolithiasis | 0 | 0 | 0 | 1 | 0 | 0 | 1 | 0 |
| (PT) | (0.0) | (0.0) | (0.0) | (2.3) | (0.0) | (0.0) | (0.4) | (0.0) |
| Chronic | 0 | 0 | 0 | 1 | 1 | 0 | 2 | 0 |
| spontaneous | (0.0) | (0.0) | (0.0) | (2.3) | (2.3) | (0.0) | (0.7) | (0.0) |
| urticaria | | | | | | | | |
| (PT) | | | | | | | | |
| Patients | 29 | 23 | 27 | 21 | 26 | 29 | 155 | 18 |
| with at | (65.9) | (52.3) | (57.4) | (47.7) | (60.5) | (64.4) | (58.1) | (42.9) |
| least one | | | | | | | | |
| AE | | | | | | | | |
| Mild | 22 | 16 | 18 | 9 | 16 | 22 | 103 | 14 |
| | (50.0) | (36.4) | (38.3) | (20.5) | (37.2) | (48.9) | (38.6) | (33.3) |
| Moderate | 7 | 6 | 8 | 10 | 9 | 5 | 45 | 4 |
| | (15.9) | (13.6) | (17.0) | (22.7) | (20.9) | (11.1) | (16.9) | (9.5) |
| Severe | 0 | 1 | 1 | 2 | 1 | 2 | 7 | 0 |
| | (0.0) | (2.3) | (2.1) | (4.5) | (2.3) | (4.4) | (2.6) | (0.0) |

AEs by preferred term (≥10% in any treatment group)

| | | | | | | | | |
|---|---|---|---|---|---|---|---|---|
| Headache | 1 | 7 | 4 | 3 | 6 | 5 | 26 | 6 |
| | (2.3) | (15.9) | (8.5) | (6.8) | (14.0) | (11.1) | (9.7) | (14.3) |
| Nasopharyngitis | 7 | 2 | 2 | 4 | 4 | 4 | 23 | 3 |
| | (15.9) | (4.5) | (4.3) | (9.1) | (9.3) | (8.9) | (8.6) | (7.1) |
| Discontinued | 0 | 0 | 0 | 3 | 1 | 3 | 7 | 0 |
| study | (0.0) | (0.0) | (0.0) | (6.8) | (2.3) | (6.7) | (2.6) | (0.0) |
| treatment | | | | | | | | |
| due to | | | | | | | | |
| AE(s) | | | | | | | | |

AEs by primary system organ class

| | | | | | | | | |
|---|---|---|---|---|---|---|---|---|
| Infections | 12 | 9 | 14 | 6 | 12 | 11 | 64 | 9 |
| and | (27.3) | (20.5) | (29.8) | (13.6) | (27.9) | (24.4) | (24.0) | (21.4) |
| infestations | | | | | | | | |
| Skin and | 7 | 9 | 5 | 6 | 12 | 6 | 45 | 2 |
| subcutaneous | (15.9) | (20.5) | (10.6) | (13.6) | (27.9) | (13.3) | (16.9) | (4.8) |
| tissue | | | | | | | | |
| disorders | | | | | | | | |
| Nervous | 3 | 10 | 7 | 4 | 6 | 5 | 35 | 7 |
| system | (6.8) | (22.7) | (14.9) | (9.1) | (14.0) | (11.1) | (13.1) | (16.7) |
| disorders | | | | | | | | |
| Gastrointestinal | 7 | 4 | 6 | 6 | 2 | 5 | 30 | 5 |
| disorders | (15.9) | (9.1) | (12.8) | (13.6) | (4.7) | (11.1) | (11.2) | (11.9) |
| Musculoskeletal | 4 | 1 | 3 | 3 | 7 | 6 | 24 | 2 |
| and connective | (9.1) | (2.3) | (6.4) | (6.8) | (16.3) | (13.3) | (9.0) | (4.8) |
| tissue | | | | | | | | |
| disorders | | | | | | | | |
| Investigations | 6 | 4 | 6 | 2 | 4 | 3 | 25 | 3 |
| | (13.6) | (9.1) | (12.8) | (4.5) | (9.3) | (6.7) | (9.4) | (7.1) |

AE, adverse event; b.i.d., two times a day; N, number of patients; PT, preferred term; q.d., once a day; SAE, serious adverse event; SOC, system organ class MedDRA version 24.0 was used for reporting.

### Summary of Safety in Phase 2b Study (Extension Phase) in CSU Subjects (Interim Results)

In a 52-week open label extension study to evaluate the long-term safety and tolerability of LOU064 in eligible subjects with CSU who participated in the Phase 2b study the dose used was 100 mg b.i.d.

No safety signal has been observed based on an interim analysis of the 100 subjects who received at least 1 dose of LOU064 with a median exposure of 17.86 weeks (range: 2.9 weeks to 44.7). At the time of cut-off, 93 subjects (93%) were ongoing, and 7 subjects had discontinued from the study; none of the discontinuations were due to adverse events. Table 4 presents the safety summary observed in the Phase 2b study up to the cut-off date for the interim analysis.

TABLE 4
Interim analysis for open label extension study: Deaths,
other serious or clinically significant adverse events
or related discontinuations (Safety Set)

| | LOU064 | |
|---|---|---|
| | 100 mg B.I.D. | |
| | N = 100 | |
| | n (%) | |
| Patients with AE(s) | 58 | (58) |
| Patients with serious or other significant events | | |
| Death | 0 | |
| Non-fatal SAE | 3 | (3.0) |
| Discontinued study due to any AE(s) | 0 | |
| Discontinued study due to an SAE(s) | 0 | |
| Treatment interruption due to AE(s) | 5 | (5.0) |
| Treatment interruption due to SAE(s) | 1 | (1.0) |

Interim analysis cut off 31 Aug. 2020

Fifty-eight subjects (58%) experienced at least one treatment emergent AE. The majority of AEs were non-serious, did not lead to treatment discontinuation and were mild in severity. The most frequently affected SOC were Infections and infestations (14%) followed by Skin and subcutaneous tissue disorders (13%) with no trends with respect to specific adverse events. The most common adverse event preferred terms (≥2%) were headache (6%), diarrhea (4%), dizziness (3%) and gastroenteritis (3%); no bleeding events (defined as events under Haemorrhages SMQ broad and the PTs including Platelet aggregation abnormal, Platelet aggregation decreased, Platelet aggregation inhibition, Platelet dysfunction, Platelet function test abnormal and Platelet toxicity) or events under SOC Blood and lymphatic system disorders were reported. Three SAEs were reported: ovarian cyst, chest pain and appendicitis; none were considered related to study drug.

Conclusions from the Phase 2b Study and Corresponding Open Label Extension Study

Taken together, there have been no new or unexpected safety findings in the Phase 2b study, across all the doses assessed. Furthermore, in the corresponding CSU extension study, which uses LOU064 100 mg b.i.d. open-label, no safety signals have been observed in 100 subjects enrolled as of 31 Aug. 2020. The proposed dose of 100 mg LOU064 b.i.d. is considered to be well tolerated and with a favorable safety profile.

Summary of Safety in Phase 2b Study (Extension Phase) in CSU Subjects (Interim Results/Patients with Medium Exposure of 35.14 Weeks)

In the above 52-week open label extension study to evaluate the long-term safety and tolerability of LOU064 in eligible subjects with CSU who participated in the Phase 2b study with a dose of 100 mg b.i.d., a new interim analysis was performed with patients (N=183) with a medium exposure of 35.14 weeks and the results were compared to the safety results in the randomized double-blind, placebo-controlled Ph2b core study, in adult patients with CSU who received (1:1:1:1:1:1:1) remibrutinib 10 mg qd (once daily), 35 mg qd, 100 mg qd, 10 mg bid (twice daily), 25 mg bid, or 100 mg bid or placebo up to 12 weeks (wks) (NCT03926611). (Table 5)

In the long-term exposure of the ES (median 35.14 wks, N=183), the proportion of patients with at least one adverse effect (AE) on remibrutinib treatment (57.4% [n=105]) was similar to the CS (presented through for any remibrutinib dose) (58.1% [n=155]; median 12.14 wks, N=267). In the ES, there were 4 serious adverse effects (SAEs), 6 AEs leading to treatment discontinuation and no deaths. The incidences of AEs by primary organ class (SOC) reported in the ES and CS were similar: infections and infestations (23.0% and 24.0%), followed by skin/subcutaneous tissue disorder (17.5% and 16.9%) (Table 5). Incidences of reported AEs by preferred term were comparable in ES and CS with headache (6.6% and 9.7%) being most common. Incidence of AESI in the ES such as infections (23%), bleeding (4.4%) and cytopenias (0.5%) were in line with the CS. Newly occurring notable transaminase increases were single in both ES (isolated ALT>3xULN, normalized within 4 weeks, in 1 patient discontinued early for personal reasons) and CS (ALT>5xULN in 1 patient, normalised on treatment). The analysis of laboratory parameters did not reveal significant safety concerns and no clinically meaningful changes in vital signs were observed. There were no significant ECG findings or QT of >500 ms noted in any patient.

### CONCLUSION

Remibrutinib showed a favorable safety profile across the whole dose range with no new safety signals observed over longer-term exposure to 100 mg bid dose up to 52 wks in patients with CSU.

TABLE 5
Safety profile of remibrutinib(LOU064) in Phase 2b core and extension study (safety set)

| Core study (CS) | | | | | | | | | ES |
|---|---|---|---|---|---|---|---|---|---|
| | LOU064 | | | | | | | | LOU064 |
| | 10 mg | 35 mg | 100 mg | 10 mg | 25 mg | 100 mg | Any | | 100 mg |
| Patients | q.d. | q.d. | q.d. | b.i.d. | b.i.d. | b.i.d. | dose | Placebo | b.i.d. |
| n (%) | N = 44 | N = 44 | N = 47 | N = 44 | N = 43 | N = 45 | N = 267 | N = 42 | (N = 183) |
| Patients | 29 | 23 | 27 | 21 | 26 | 29 | 155 | 18 | 105 |
| with ≥1 AE | (65.9) | (52.3) | (57.4) | (47.7) | (60.5) | (64.4) | (58.1) | (42.9) | (57.4) |
| Discontinued | 0 | 0 | 0 | 3 | 1 | 3 | 7 | 0 | 6 |
| study | (0.0) | (0.0) | (0.0) | (6.8) | (2.3) | (6.7) | (2.6) | (0.0) | (3.3) |
| treatment | | | | | | | | | |
| due to | | | | | | | | | |
| AE(s) | | | | | | | | | |
| Patients | 1 | 0 | 0 | 2 | 2 | 0 | 5 | 0 | 4 |
| with | (2.3) | (0.0) | (0.0) | (4.5) | (4.7) | (0.0) | (1.9) | (0.0) | (2.2) |
| SAE(s) | | | | | | | | | |

Most frequent AEs by primary system organ class (≥10% of all patients receiving
any LOU064 dose or placebo group in CS or in 100 mg b.i.d. in ES)

| | | | | | | | | | |
|---|---|---|---|---|---|---|---|---|---|
| Infections | 12 | 9 | 14 | 6 | 12 | 11 | 64 | 9 | 42 |
| and | (27.3) | (20.5) | (29.8) | (13.6) | (27.9) | (24.4) | (24.0) | (21.4) | (23.0) |
| infestations | | | | | | | | | |
| Skin and | 7 | 9 | 5 | 6 | 12 | 6 | 45 | 2 | 32 |
| subcutaneous | (15.9) | (20.5) | (10.6) | (13.6) | (27.9) | (13.3) | (16.9) | (4.8) | (17.5) |
| tissue | | | | | | | | | |
| disorders | | | | | | | | | |
| Nervous | 3 | 10 | 7 | 4 | 6 | 5 | 35 | 7 | 19 |
| system | (6.8) | (22.7) | (14.9) | (9.1) | (14.0) | (11.1) | (13.1) | (16.7) | (10.4) |
| disorders | | | | | | | | | |
| Gastrointestinal | 7 | 4 | 6 | 6 | 2 | 5 | 30 | 5 | 26 |
| diseases | (15.9) | (9.1) | (12.8) | (13.6) | (4.7) | (11.1) | (11.2) | (11.9) | (14.2) |

Most frequent AEs by PT (≥5% of patients in dose
or placebo group in CS or in 100 mg b.i.d. in ES)

| | | | | | | | | | |
|---|---|---|---|---|---|---|---|---|---|
| Headache | 1 | 7 | 4 | 3 | 6 | 5 | 26 | 6 | 12 |
| | (2.3) | (15.9) | (8.5) | (6.8) | (14.0) | (11.1) | (9.7) | (14.3) | (6.6) |
| Nasopharyngitis | 7 | 2 | 2 | 4 | 4 | 4 | 23 | 3 | 6 |
| | (15.9) | (4.5) | (4.3) | (9.1) | (9.3) | (8.9) | (8.6) | (7.1) | (3.3) |
| Chronic | 3 | 2 | 3 | 4 | 2 | 2 | 16 | 1 | 6 |
| spontaneous | (6.8%) | (4.5%) | (6.4%) | (9.1%) | (4.7%) | (4.4%) | (6.0%) | (2.4%) | (3.3) |
| urticaria | | | | | | | | | |

### Example 5

Below a preferred pharmaceutical composition (film-coated tablet) is illustrated.

| | Amount per 100 mg | |
|---|---|---|
| | Film-coated tablet | |
| Ingredient | (mg) | Function |
| Tablet core | | |
| LOU064 | 100.0 | Drug substance |
| Mannitol | 243.8 | Carrier |
| Cellulose, microcrystalline/ | 85.8 | Diluent |
| Microcrystalline Cellulose | | |
| Copovidone | 50.0 | Binder |
| Croscarmellose sodium | 31.2 | Disintegrant |
| Sodium Stearyl Fumarate | 5.2 | Lubricant |
| Sodium laurilsulfate/Sodium | 4.0 | Surfactant |
| lauryl sulfate | | |
| Water, purified/Purified water¹ | — | Suspending |
| | | agent/Solvent |
| Core tablet weight | 520.0 | |
| Coating | | |
| Basic coating premix, yellow | 14.2 | Film-coating |
| Basic coating premix, red | 4.4 | Film-coating |
| Basic coating premix, white | 4.4 | Film-coating |
| Basic coating premix, black | 1.2 | Film-coating |
| Water, purified² | — | Granulation |
| | | liquid |
| Total film-coated tablet weight | 544.2 | |

^{1,2}Removed during processing

### Example 6

Below another preferred pharmaceutical composition (hard gelatin capsule) is illustrated.

| | Amount per 50 mg | |
|---|---|---|
| Ingredient | capsule (mg) | Function |
| Capsule fill | | |
| LOU064 | 50.000 | Drug substance |
| Mannitol | 147.500 | Carrier |
| Cellulose, microcrystalline/ | 78.100 | Diluent |
| Microcrystalline Cellulose | | |
| Copovidone | 50.000 | Binder |
| Sodium hydrogen carbonate/ | 10.200 | Disintegrant |
| Sodium bicarbonate | | |
| Magnesium stearate ¹ | 1.700 | Lubricant |
| Sodium laurilsulfate/Sodium | 2.500 | Surfactant |
| lauryl sulfate | | |
| Water, purified/Purified water ² | — | Suspending |
| | | agent/Solvent |
| Capsule fill weight | 340.000 | |
| Empty capsule shell | | |
| Capsule shell | 96.000 | |
| (theoretical weight) ³ | | |
| Total capsule weight | 436.000 | |
| Components of | | |
| one capsule shell | | |

| Capsule shell components ¹ | Reference to standards |
|---|---|
| Gelatin | Ph. Eur., USP/NF |
| Titanium dioxide (E 171) | Ph. Eur., USP/NF |
| Iron oxide (E172), | Regulation (EU) |
| red/Ferric oxide | 231/2012 ², USP/NF |

¹ For both cap and body
² Commission regulation (EU) laying down specifications for food additives
E = Official number used in the European Union for colorants
The hard gelatin capsules show good stability, and the current shelf-life is 36 months.

### Example 7

### Prediction of BTK Occupancy Using a Translational PK/PD Model for LOU064

BTK occupancy in blood is not an informative biomarker for the purpose of dose selection due to LOU064 pharmacological properties (irreversible binding). It reaches full occupancy even at low doses before showing pharmacological activity through other biomarkers (CD63, CD203c, skin-prick test). Occupancy in tissue may be more representative of the efficacy of LOU064.

### Objectives

The purpose of this analysis was to characterize the pharmacokinetics (PK) of LOU064 in healthy volunteers and to use a previously developed, translational target occupancy model to simulate BTK occupancy in human spleen/tissues across a range of doses and dosing regimen (BID vs QD).

### Data

Pharmacokinetic data from a Phase I clinical study reported by Kaul et al. (2021) were used in the current analysis, including 102 patients.

### Methods

A translational target occupancy model to simulate BTK occupancy in spleen/tissues was developed using a two-step approach.

In a first step, a population PK model was established to describe LOU064 PK data from Phase I clinical study reported by Kaul et al. (2021). In a second step, the parameter estimates from the population PK model were used in the BTK occupancy model to predict BTK occupancy in blood and spleen/tissues. Ultimately the BTK occupancy model was used to predict the BTK occupancy in spleen/tissues for different dosing regimens (QD, BID) at different doses.

### Results

A population PK model has been developed to describe the interim PK from a Phase I clinical study reported by Kaul et al. (2021). In order to address the change in clearance after repeated dosing for doses lower than 50 mg (lower clearance at steady state at Day 12 when compared to Day 1 with no difference at higher doses), the clearance was modeled as a function of exponential time decay for doses less than 50 mg and a constant clearance for doses above 50 mg. Overall the resulting population model described the PK data reasonably well.

The PK parameter estimates were used in a translational BTK occupancy model to simulate BTK occupancy at steady state. The BTK occupancy simulations showed that BID dosing is more effective than QD dosing at the same dose to achieve higher BTK occupancy (at trough or averaged over 24-hour interval).

For a selected number of doses at QD and BID regimen, steady-state BTK occupancy at trough and averaged over a period of 24 hours are shown in FIG. 21A (Trough over 24 hours of BTK Occupancy at steady state) and FIG. 21B (Average over 24 hours of BTK Occupancy at steady state), respectively for dosing regimens of 10 mg, 35 mg, 100 mg once daily and 10 mg, 25 mg and 100 mg twice daily. Both figures show that a daily dose up to 200 mg (100 mg BID) may be required to achieve a trough BTK occupancy ≥80% in a peripheral target tissue.

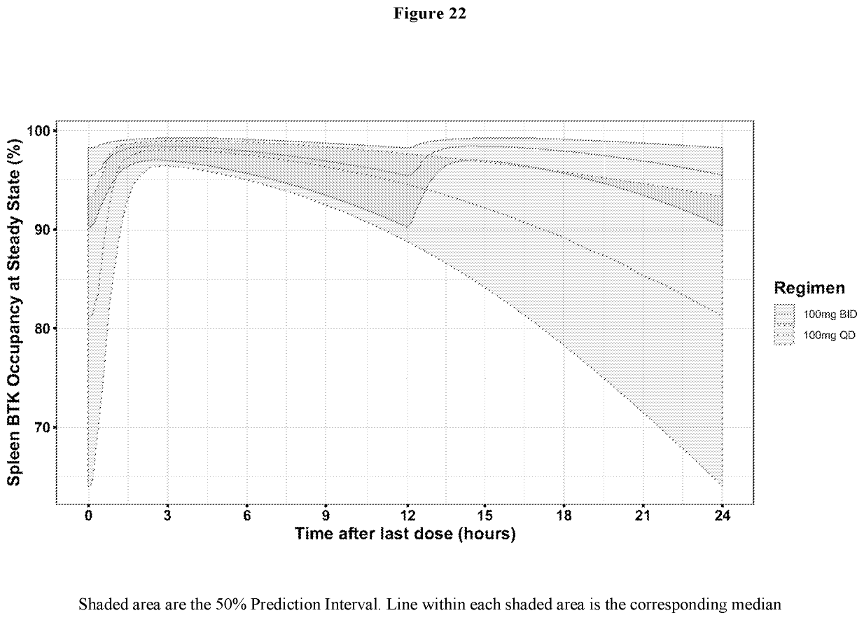

Simulations were performed to compare different dosing regimens. A comparison of simulated spleen BTK occupancy at steady state of 100 mg BID vs. 100 mg QD over time is shown in FIG. 22. The graph shows that the occupancy from BID dosing is higher and less variable compared to QD dosing.

### Conclusions:

The BTK occupancy simulations showed that BID dosing is more effective than QD dosing at the same dose to achieve higher BTK occupancy (at trough or averaged over 24-hour interval).

### Example 8

Phase III Clinical Studies Showing the Efficacy and Safety of LOU064 in the Treatment of Patients with RMS

### Objective

To demonstrate that LOU064 (100 mg p.o. b.i.d.) is superior to teriflunomide (14 mg p.o. once daily) in reducing the frequency of confirmed relapses as evaluated by the annualized relapse rate (ARR) in subjects with relapsing MS multiple sclerosis patients are treated with LOU064.

### Methods

Two identical double-blind, randomized, double-dummy, active comparator-controlled, parallel-group, multi-center studies are performed assessing the efficacy and safety of LOU064 (100 mg b.i.d.) vs. teriflunomide (14 mg) in the treatment of patients with RMS plus their long-term open label safety extension. In total, approximately 1600 subjects (800 per study) participate in these Phase III studies.

Patients are randomised (1:1) to receive either LOU064 100 mg p.o. b.i.d. or teriflunomide 14 mg orally once daily, for up to 30 months, starting from Day 1. The studies have flexible durations, with termination occurring in the blinded core treatment epoch according to pre-specified criteria. Patients aged 18-55 years, diagnosed with relapsing MS according to the 2017 McDonald diagnostic criteria (this would include relapsing-remitting course (RRMS) or secondary progressive (SPMS) course with disease activity), with an Expanded Disability Status Scale (EDSS) score (according to Kurtzke, Neurology: 1983, Nov; 33(11): 1444-52) of 0-5.5 at screening who experienced ≥1 relapse in the past year or ≥2 relapses in the past 2 years or a positive gadolinium-enhancing (Gd+) MRI scan during the 6 months before randomization and who were neurologically stable within 1 month prior to randomization were included.

The primary endpoint of the study is the annualized relapse rate (ARR), i.e. the number of confirmed relapses per year. Main secondary endpoints include disability endpoints (pooled CDP, i.e. time to disability progression as measured by 3-month confirmed disability progression (3mCDP) and 6-month confirmed progression (6mCDP) on EDSS based on the pooled data of two studies), MRI-endpoints (i.e. number of T1 gadolinium (Gd)-enhancing lesions per MRI scan, number of new or enlarging T2 lesions on MRI per year (annualized T2 lesion rate)), neurofilament light chain (NfL) concentration in serum, no evidence of disease activity-3 (NEDA3) based on the pooled data of two studies, and rate of brain volume loss (BVL) based on assessments of percentage brain volume change from baseline.

ARR and MRI endpoints are analyzed using the NB model; CDP endpoints are analyzed using the Cox regression model.

A total of approximately 800 subjects is randomized to the study drug in a 1:1 ratio (400 per treatment arm) for each study, providing >90% power to demonstrate superiority of LOU064 over teriflunomide for the primary endpoint (ARR) in each study assuming a 40% relative reduction in ARR by remibrutinib (LOU064) based on a 1-sided test with a 0.025 significance level and a 20% dropout rate in 2 years. A combined 1600 patients from the two studies provides 90% power to demonstrate superiority of LOU064 over teriflunomide in 3mCDP assuming 40% risk reduction based on a 1-sided test with 0.025 significance level and 20% dropout rate in 2 years. Additionally efficacy of LOU064 (100 mg bid) as well as superiority of LOU064 treatment as compared to Teriflunomide (14 mg QD) in treating RMS patients is also assessed by measuring secondary endpoints such as SDMT, T25 FW and 9HPT as well as measuring patient reported outcome such as MSIS-29, HUI-IL, PHQ-9, GAD-7, FSIQ-RMS and BPI-SF)

A summary of the secondary outcome measures is presented in table below:

| Outcome measure | Timeframe | description |
|---|---|---|
| Time to 3-month | Baseline, every 3 | lime to 3-month confirmed disability progression |
| confirmed disability | months, up to 2.5 | (3mCDP) is defined as an increase from baseline in |
| progression (3mCDP) on | years | Expanded Disability Status Scale (EDSS) sustained |
| Expanded Disability | | for at least 3 months |
| Status Scale (EDSS) - | | |
| Core | | |
| Time to 6-month | Baseline, every 6 | Time to 6-month confirmed disability progression |
| confirmed disability | months, up to 2.5 | (6mCDP) is defined as an increase from baseline in |
| progression (6mCDP) on | years | Expanded Disability Status Scale (EDSS) sustained for |
| EDSS - Core | | at least 6 months. |
| Annualized rate of new | Baseline up to | Number of new/enlarging T2 lesions compared to las |
| or enlarging 2 lesion- | 2.5 years | available MRI scan at various points |
| core | | |
| Neurofilament light | Baseline, Months | Reduction of neuronal damage defined as Nfl |
| chain (Nfl) | 3, 6, 12, 24, 30 | concentration in serum |
| concentration in serum- | | |
| core | | |
| Number of Gd- | Baseline up to | Total number of Gd-enhancing T1 lesions across all |
| enhancing T1 lesions | 2.5 years | scans per patient adjusted for different number of |
| per MRI scan - Core | | scans due to variable follow-up time in study. |
| Percentage of | Baseline, every 6 | Disease-Activity-free status based on Percentage of |
| participants with No | months, up to 2.5 | participants with No Evidence of Disease Activity-3 |
| Evidence of Disease | years. | (NEDA-3), as assessed by absence of confirmed MS |
| Activity-3 (NEDA-3) - | | relapses, 6mCDP and new/enlarging T2 lesions on MRI |
| Core | | |
| Time to first confirmed | Baseline up to | A 6-months confirmed disability improvement |
| relapse - core | 2.5 years | (6mCDI) sustained until end of the study |
| Change from baseline in | Baseline up to | Symbol Digit Modalities Test (SDMT) measures |
| the Symbol Digit | 2.5 years | processing speed. The score is the number of correctly |
| Modalities Test (SDMT) | | coded items in 90 seconds. (max = 110, min = 0). Higher |
| (pooled data) - Core | | scores indicate improvement. |
| | | Lower scores indicate worsening |
| Time to 6-month confirmed | Baseline, every 6 | The patient is assessed of the walking speed to cover 25 |
| worsening by atleast 20% | months, up to 2.5 | foot distance. The time is recorded in seconds. Longer time |
| in the Timed 25-foot | years | indicates poorer upper limb function. 20% improvement is |
| walk test (T25FW) - | | defined as 20% shorter time in seconds. |
| Core | | |
| Time to 6-month | Baseline, every 6 | 9 Hole Peg Test is a test of upper limb function. |
| confirmed worsening by | months up to 2.5 | Longer time indicates poorer upper limb function. |
| at least 20% in the | years | 20% improvement is defined as 20% shorter time in |
| Timed 9-hole peg test | | seconds. |
| (9HPT)(pooled data) - | | |
| Core | | |
| Time to composite 6- | Baseline, every 6 | Time to composite is evaluated by 6mCDP or 6-month |
| month confirmed | months up to 2.5 | confirmed worsening by at least 20% in T25FW or 9HPT. |
| disability progression- | years | |
| core | | |
| Change from baseline in | Baseline. Months | Percent change from baseline in total T2 lesion volume |
| T2 lesions volume- core | 6, 12, 24 and 30 | |
| Change from baseline in | Baseline up to | 20-item, self-administered questionnaire organized with |
| Fatigue Symptoms and | 2.5 years | two symptom domains (energy, muscle weakness) and seven |
| Impacts Questionnaire - | | impact domains (daily activities, cognition, emotions, |
| Relapsing Multiple | | physical impact, self-care, sleep and social impact). |
| Sclerosis (FSIQ-RMS) - | | Global score ranges from 0 to 100 where higher score |
| Core | | represents greater fatigue |
| Change from baseline in | Baseline up to | 7-item, self-administered scale. |
| Generalized Anxiety | 2.5 years | Response options for the scale consist of a 4- point Likert |
| Disorder Scale (GAD- | | Scale: 0: not at all; 1: several days; 2: more than half |
| 7)- Core | | the days; 3: nearly every day. It has a global score |
| | | ranging from 0-21. |
| | | Higher score means higher severity of anxiety symptoms. |
| Change from baseline in | Baseline up to | 9-item, self-administered scale. Scores can range from |
| Patient Health | 2.5 years | 0 to 27, since each of the 9 items can score from 0 |
| Questionnaire-9 (PHQ- | | (not at all) to 3 (nearly every day). PHQ-9 scores of 5, |
| 9) - Core | | 10, 15, and 20 represented mild, moderate, moderately |
| | | severe, and severe depression, respectively. |
| Change from baseline in | Baseline up to | 15-item, self-administered questionnaire to assess the |
| Brief Pain Inventory- | 2.5 years | severity of pain and the impact of pain on daily |
| short form (BPI-SF) - | | functions. Includes 7-item interference scale. It has a |
| Core | | 10 point response optio, ranging from 0 (does not |
| | | interefere) to 10 (completely interferes). Global score |
| | | ranges from 0 to 10, where lower scores represent |
| | | lower pain. |
| Change from baseline in | Baseline up to | MSIS-29 is a 29-item, self-administered questionnaire |
| Multiple Sclerosis | 2.5 years | that includes 2 domains, physical and psychological. |
| Impact Scale (MSIS-29) - | | Responses are captured on a 4-point scale ranging |
| Core | | from “not at all” (1) to “extremely” (4), where higher |
| | | scores reflect greater impact on day to day life. |
| Number and percentage | Baseline up to | Self-reported questionnaire that assesses Suicidal |
| of participants with | 2.5 years | Ideation and Suicidal Behavior |
| suicidal ideation and/or | | |
| suicidal behavior - Core | | |
| Number of adverse | Baseline up to | Adverse events will be collected throughout the trial. |
| events and serious | 2.5 years | Any safety assessment findings such as laboratory, |
| adversed events- core | | vital signs, electrocardiogram data that are considered |
| | | clinically significant or meet the protocol definition of |
| | | an adverse event will be reported as adverse event or |
| | | serious adverse event, as appropriate |

An extension part is run to measure long term safety and efficacy of LOU064 (100 mg bid).

The extension part is an open-label, single arm, fixed dose design in which eligible participants are treated with LOU064 up to 5 years. Participants on LOU064 in core study (up to 30 months) will continue on LOU064 (100 mg bid) and participants on teriflunomide (14 mg QD) in core study will switch to LOU064 (100 mg bid)

| | | |
|---|---|---|
| Number of adverse events | Day 1 Extension | Adverse events will be collected throughout the trial. |
| and serious adverse | up to 5 years | Any safety assessment findings such as laboratory, |
| events - Extension | | vital signs, electrocardiogram data that are considered |
| | | clinically significant or meet the protocol definition of |
| | | an adverse event will be reported as adverse event or |
| | | serious adverse event, as appropriate |
| Annualized relapse rate | Day 1 Extension | A confirmed relapse is defined as a clinically |
| (ARR) of confirmed | up to 5 years | relevant change in the Expanded Disability Status |
| relapses - Extension | | Scale (EDSS). ARR is defined as the number of |
| | | confirmed MS relapses in a year. |
| Annualized rate of new or | Day 1 Extension | Number of new/enlarging T2 lesions compared to last |
| enlarging T2 lesion - | up to 5 years | available MRI scan at various time points. |
| Extension | | |
| Time to 6-month | Day 1 Extension, | Time to 6-month confirmed disability progression |
| confirmed disability | every 6 months, | (6mCDP) is defined as an increase from baseline in |
| progression (6mCDP) | up to 5 years | Expanded Disability Status Scale (EDSS) sustained for |
| on EDSS - Extension | | at least 6 months. |
| Change from baseline | Day 1 Extension | Symbol Digit Modalities Test (SDMT) measures |
| in the Symbol Digit | up to 5 years | processing speed. The score is the number of correctly |
| Modalities Test | | coded items in 90 seconds. (max = 110, min = O). Higher |
| (SDMT) (pooled | | scores indicate improvement. |
| data) - Extension | | Lower scores indicate worsening |
| Neurofilament light chain | Day 1 Extension | Reduction of neuronal damage defined as |
| (NIL) concentration in | up to 5 years | Neurofilament light chain (NIL) concentration in serum |
| serum - Extension | | |
| Change from baseline in | Day 1 Extension | 20-item, self-administered questionnaire organized |
| Fatigue Symptoms and | up to 5 years | with two symptom domains (energy, muscle |
| Impacts Questionnaire - | | weakness) and seven impact domains (daily activities, |
| Relapsing Multiple | | cognition, emotions, physical impact, self-care, sleep |
| Sclerosis (FSIQ-RMS) - | | and social impact). Global score ranges from 0 to 100 |
| Extension | | where higher score represent greater fatigue |
| Change from baseline in | Day 1 Extension | 7-item, self-administered scale. |
| Generalized Anxiety | up to 5 years | Response options for the scale consist of a 4- point |
| Disorder Scale (GAD-7)- | | Likert Scale: 0: not at all; 1: several days; 2: more than |
| Extension | | half the days; 3: nearly every day. It has a global score |
| | | ranging from 0-21. |
| | | Higher score means higher severity of anxiety symptoms. |
| Change from baseline in | Day 1 Extension | 9-item, self-administered scale. Scores can range from |
| Patient Health | up to 5 years | Oto 27, since each of the 9 items can be scored from O |
| Questionnaire-9 | | (not at all) to 3 (nearly every day). |
| (PHQ-9) - Extension | | PHQ-9 scores of 5, 10, 15, and 20 represented mild, |
| | | moderate, moderately severe, and severe depression, |
| | | respectively. |
| Change from baseline in | Day 1 Extension | 15-item, self-administered questionnaire to assess the |
| Brief Pain Inventory - | up to 5 years | severity of pain and the impact of pain on daily |
| short form (BPI-SF) - | | functions. Includes seven-item interference scale |
| Extension | | It has a 10-point response option, ranging from 0 (does |
| | | not interfere) to 10 (completely interferes). Global |
| | | score ranges from Oto 10, where lower scores |
| | | represent lower pain. |
| Change from baseline in | Day 1 Extension | The HUI-Ill is a 17-item, self-administered index that |
| Health Utilities Index | up to 5 years | measures eight health-related quality of life areas |
| (HUI-Ill) - Extension | | including vision, hearing, speech, ambulation/mobility |
| | | pain, dexterity, emotion, and cognition. |
| Change from baseline | Day 1 Extension | MSIS-29 is a 29-item, self-administered questionnaire |
| in Multiple Sclerosis | up to 5 years | that includes 2 domains, physical and psychological. |
| Impact Scale (MSIS- | | Responses are captured on a 4-point scale ranging |
| 29) - Extension | | from “not at all” (1) to “extremely” (4), where higher |
| | | scores reflect greater impact on day to day life. |

### Example 9

scRNA-Seq Analysis of RatMOG EAE Mice Treated with LOU064

### Methods

Processing and Quality Control of scRNA-Seq Data

10X Genomics Chromium raw sequencing reads were processed with Cell Ranger [Zheng, G. et al. (2017), Nat Commun 8, 14049]. All subsequent analyses were carried out in R v4.1 and Bioconductor v3.14, loosely following the guidelines in “Orchestrating single-cell analysis with Bioconductor” [Amezquita, R. A. et al (2020), Nat Methods 17, 137-145] and using the framework provided by the scuttle, scater [Davis J McCarthy et al. (2017), Bioinformatics, Vol 33, 8, 1179-1186] and scran [Lun ATL et al. (2016), F1000 Research, 5:2122;

https://doi.org/10.12688/fl000research.9501.2] packages. One sample was excluded from the analysis due to the low number of UMIs per cell and suspected contamination with ambient RNA. Adaptive-threshold quality control was used to remove low-quality cells with low library size, low number of detected genes, or high proportion of mitochondrial reads. Doublets were identified and removed using scDblFinder [Germain P L et al. (2021) F1OOOResearch 10:979;

https://doi.org/10.12688/fl000research.73600.1]. In total, 76287 cells passed quality control.

### Normalization, Feature Selection and Dimensionality Reduction

To correct for library size and composition biases, normalization was performed by pooling cells and deconvoluting size factors, followed by a log2 transformation. The per-gene mean—variance relationship was modelled separately for the two tissues. Highly variable genes were selected using a 5% FDR threshold under the null hypothesis that the biological component of the gene variation is equal to zero. These genes were then used to perform a principal component analysis and only the PCs that related to the biological component of the gene variation were retained. Finally, the selected PCs were used to obtain a reduced dimensionality representation of the data (UMAP).

### Clustering and Cell Type Annotation

A shared nearest neighbour graph-based clustering approach with 5 nearest neighbours, Jaccard index as the weighting scheme, and Louvain as the clustering method was used to identify clusters of similar cells. Cell clusters were assigned to cell types using SingleR [Aran D et al. (2019), Nat Immunol. 20, 163-172] and two sets of bulk transcriptomics references of mouse cell types [Heng T S et al. (2008), Nat. Immunol. 9, 1091-1094; Benayoun B et al. (2019), Genone Res. 29, 697-709]. Microglia were further annotated as homeostatic microglia (HM) and disease-associated microglia (DAM) using UCell [Andreatta M. et al. (2021), Comput Struct Biotechnol J. 19:3796-3798] and the marker genes in [Deczkowska A et al. (2018), Cell. 17; 173(5):1073-1081].

### Differential Expression and Enrichment Analyses

For each cell type, pseudobulk differential expression analysis was performed using edgeR [Robinson M D et al. (2010), Bioinformatics, 26(1), 139-140], followed by gene set enrichment analysis with fgsea [Gennady Korotkevich et al., doi: https://doi.org/10.1101/060012] and multiple gene set collection from MSigDB [Arthur Liberson et al., Bioinformatics, Vol 27, 12:1739-1740]. A neuroinflammation signature from the Human Phenotype Ontology [Kohler S. et al. (2021) Nucleic Acids Res. 49(D1):D1207-D1217. doi: 10.1093/nar/gkaa1043] was scored with UCell [Andreatta M. et al. (2021), Comput Struct Biotechnol J. 19:3796-3798] and its significance assessed with a one-tailed Mann-Whitney U test.

### Results

Inhibition of Btk in RatMOG EAE Mice with LOU064 Dampens Neuroinflammation in Microglial Cells

By profiling brains and spinal cords of RatMOG EAE mice treated with LOU064 or normal food using scRNA-seq, we identified 13 different cell types, including stromal cells (fibroblasts, endothelial cells), all major immune cell types recruited in the CNS (B cells, T cells, DCs, monocytes and macrophages), and resident cells of the CNS: neurons, neuroepithelial cells, astrocytes, oligodendrocytes and microglia, which we further classified in HM and DAM.

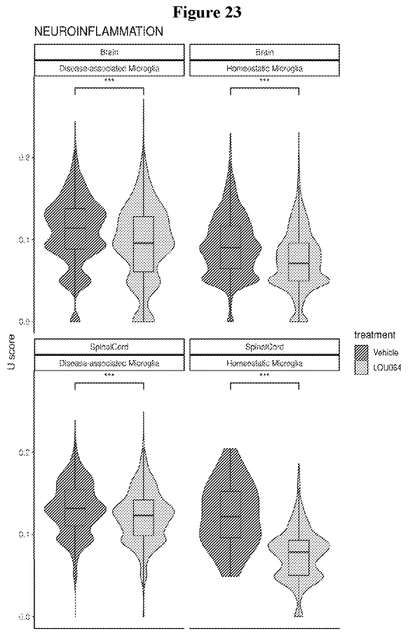

Following the identification of genes differentially expressed between LOU064-treated animals and controls across the 13 cell types, we investigated whether a neuroinflammation gene signature (sourced from the Human Phenotype Ontology) was affected by LOU064 in microglia, and observed a significant downregulation across most conditions (FIG. 23).

### Example 10: Evaluation of the Modulation of Immune Response to Three Different Types of Vaccines by Concomitant and Interrupted Administration of Remibrutinib in Health Subject

### Objectives and Related Endpoints

| Objective(s) | Endpoint(s) |
|---|---|
| Primary objective(s) | Endpoint(s) for primary objective(s) |
| To evaluate non-inferiority of | Achievement of response where |
| concomitant and interrupted | response is defined as: |
| remibrutinib treatment on the immune | Influenza: >4-fold increase of |
| response following vaccinations in | anti- hemagglutinin antibody |
| healthy participants relative to placebo | titers at 28 days (Day 43) after |
| for a: | vaccination compared with |
| T cell-dependent vaccine (Seasonal | baseline (i.e. sero-conversion) |
| Influenza, quadrivalent vaccine) | PPV-23: >2-fold increase of |
| T cell-independent vaccine (PPV- | immunoglobulin G (IgG) titers |
| 23, Pneumovax ®, Merck & Co. Inc., | 28 days (Day 43) after vaccination |
| USA) | compared with baseline. |
| Secondary objective(s) | Endpoint(s) for secondary objective(s) |
| Objective(s) | Endpoint(s) |
| To assess the effect of concomitant and | T-cell dependent antibody response |
| interrupted remibrutinib treatment on | as measured by anti-KLH IgG and |
| the immune response following | IgM titers 28 days after vaccination |
| vaccinations in healthy participants | (Day 43) |
| relative to placebo for T cell-dependent | |
| de novo vaccine (KLH, Immucothel ®) | |
| To investigate the safety and tolerability | All safety assessments (including |
| of remibrutinib administered as 100 mg | vital signs, ECGs, safety laboratory |
| b.i.d. for up to 35 days in healthy | parameters, and AEs) |
| participants | |
| To explore the safety and tolerability of | All safety assessments (including |
| the vaccinations administered to healthy | vital signs, ECGs, safety laboratory |
| participants receiving remibrutinib | parameters, and AEs) |
| To assess the PK of remibrutinib at a | PK parameters: AUCtau (Day 15 |
| 100 mg b.i.d. dose | only), AUClast, Cmax, Tmax |

### Study Design

### Overall Design

This randomized, double-blind, placebo-controlled study has a parallel group design. Approximately 90 healthy female of non-childbearing potential and male participants are randomized to any of the three treatment groups in order to achieve a minimum of 72 evaluable completers considering an estimated drop-out rate up to 20%. The study will consist of a 28-day screening period, a 43-day treatment period, followed by a Study Completion evaluation (Day 57) within two weeks after last study drug administration. A safety follow-up call is performed approximately 30 days after the last study drug administration (Day 73). Participants are domiciled on Days −1 to 1 and Days 14-17. In total, the maximum study duration for each participant is about 85 days.

The impact of concomitant and interrupted remibrutinib treatment scenarios for Influenza/Pneumovax® 23 and Immucothel® is evaluated with reference to placebo.

### Study Conduct

### Screening & Baseline

Participants who meet the eligibility criteria at screening will be admitted to baseline evaluations on Day −1. All baseline safety evaluation results must be available prior to first dosing. At baseline, participants are randomized to one of the three treatment groups described below.

### Treatment

All participants receive study drug (remibrutinib 100 mg or placebo b.i.d.) from Day 1 until Day 42 and return to the clinic for End of Treatment visit at day 43. All participants also receive the quadrivalent Influenza vaccine, the PPV-23 vaccine and the KLH neoantigen vaccine on Day 15. Vaccinations should occur 3 hours after study drug administration.

During clinical visits and during domiciliation (Days −1 to 1 and Days 14-17), participants are administered the study drug by the study personnel at the clinic. Upon discharge from clinical visits during the Treatment period, study drug is provided to the participants for self-administration at home, along with the medication diary.

Safety assessments will include physical examinations, ECGs, vital signs, standard clinical laboratory evaluations (hematology, blood chemistry, urinalysis) adverse event and serious adverse event monitoring.

Multiple blood samples to assess remibrutinib pharmacokinetics will be drawn from all participants on Day 8, Day 15 and on Day 36.

### Group A (Concomitant Remibrutinib Treatment):

Participants will receive placebo (b.i.d.) from Days 1-7, followed by treatment with remibrutinib (100 mg b.i.d.) on study Days 8-15 to achieve PK/PD steady state, prior to administration of the three vaccines on Day 15. Participants will continue to receive remibrutinib (100 mg b.i.d.) until Day 42.

### Group B (Interrupted Remibrutinib Treatment):

Participants will be treated with remibrutinib 100 mg b.i.d from Day 1-7 to achieve PK/PD steady state conditions, followed by placebo (b.i.d.) administration from Day 8-28 and will be administered the three vaccines on Day 15. Treatment with remibrutinib 100 mg b.i.d. will be re-initiated treatment from Day 29 to 42.

### Group C (Placebo):

Participants in Group C will receive placebo (b.i.d) from Day 1-42 and will be vaccinated with the 3 vaccines on Day 15 under placebo conditions.

### Key Inclusion Criteria

Signed informed consent must be obtained prior to participation in the study.
Healthy, or mildly obese but otherwise healthy, male and non-childbearing potential female participants aged 18 to 55 years (inclusive).
Participants should be in good health as determined by past medical history, physical examination, vital signs, ECG, and laboratory tests at Screening and Baseline visit as indicated.
At Screening and Baseline, vital signs (systolic and diastolic blood pressure and pulse rate) will be assessed in the sitting position and again (when required by the assessment schedule) in the standing position. Sitting vital signs (after sitting 3 minutes) should be within the following ranges:
   Tympanic body temperature of 35.0 to 37.5° C.
   Systolic blood pressure (SBP) of 90 and 139 mmHg (inclusive).
   Diastolic blood pressure of (DBP) 50 and 89 mmHg (inclusive).
   Pulse rate of 45 and 90 bpm (inclusive).
Participants must weigh at least 50 kg to participate in the study and must have a body mass index (BMI) within the range of 18 to 34.9 kg/m2.
Participants must be willing to remain at the clinical site as required by the protocol and to comply with the requirements/instructions outlined in the ICF.
Able to read, speak, and understand the local language, to understand and comply with the requirements of the study.

### Key Exclusion Criteria

1. Use of other investigational drugs within 5 half-lives or 30 days prior to first dosing, whichever is longer.
2. Current evidence or past medical history of clinically significant ECG abnormalities or a family history (grandparents, parents, and siblings) of a prolonged QT interval syndrome or other abnormalities in cardiac conduction, history of additional risk factors for Torsade de Pointes (TdP) (e.g. heart failure, hypokalemia) and/or known history or current clinically significant arrhythmias. Abnormal ECG defined as PR >220 msec, QRS complex >120 msec, for males and females QTcF >450 msec, or any other morphological changes, other than early repolarization, nonspecific S-T or T-wave changes.
3. History or presence of malignancy of any organ system (other than localized basal cell carcinoma of the skin or in-situ cervical cancer), treated or untreated, within the past 5 years, regardless of whether there is evidence of local recurrence or metastases.
4. History or presence of any clinically significant disease of any major system organ class including (but not limited to) cardiovascular, pulmonary, metabolic, hepatic, renal, hematologic, endocrine, neurological or psychiatric diseases which had not resolved within two weeks prior to initial dosing
5. Hypersensitivity to remibrutinib or drugs from the same compound class or its excipients.
6. Any contraindication for the use of the Pneumovax 23, influenza or KLH vaccine including any acute infection, fever or hypersensitivity reactions or known hypersensitivity to any relevant component of the vaccines to be administered in this study (e.g., hen's egg or shellfish/KLH).
7. History of vaccination with the 2022-2023 seasonal influenza vaccine or known clinical diagnosis of influenza infection during the 2022-2023 influenza season prior to enrollment.
8. History of previous exposure or immunization with KLH.

1. A method of treating multiple sclerosis, comprising administering a therapeutically effective dose of LOU064 to a patient in need of such treatment.

2. The method according to claim 1, wherein LOU064 is administered orally at a dose of about 50 mg to about 150 mg twice daily.

3. The method according to claim 1, wherein LOU064 is administered orally at a dose of about 100 mg twice daily.

4. The method according to claim 1, wherein the treatment is a long-term treatment.

5. The method according to claim 1, wherein multiple sclerosis is selected from relapsing multiple sclerosis, in particular clinically isolated syndrome (CIS), relapsing-remitting multiple sclerosis (RRMS) and secondary progressive multiple sclerosis (SPMS), in particular active SPMS.

6. The method according to claim 1, wherein the patients are switched from a drug of an earlier disease-modifying therapy to LOU064.

7. The method according to claim 6, wherein the drug of the earlier disease-modifying therapy is selected from a B cell and/or T cell inhibitor, teriflunomide, mitoxantrone, dimethyl fumarate, cladribine, fingolimod, siponimod, ponesimod, glatiramer acetate, and interferon (e.g beta interferon).

8. The method according to claim 6, wherein the earlier disease-modifying therapy lacks efficacy.

9. The method according to claim 6, wherein the patient lacks tolerability for the earlier disease-modifying therapy.

10. The method according to claim 6, wherein the earlier disease-modifying therapy is discontinued before initiation of LOU064 administration.

11. (canceled)

12. (canceled)

13. The method according to claim 1, wherein the treatment is a monotherapy.

14-19. (canceled)

20. The method according to claim 1, wherein LOU064 is administered after a relapse.

21. The method according to claim 1, wherein LOU064 is administered after the detection of at least one Gd⁺ lesion.

22. The method according to claim 1, wherein LOU064 is administered after the detection of new or enlarging T2 lesions.

23. The method according to claim 1, wherein the patient is an adult.

24. The method according to claim 1, wherein the patient achieves at least one of the following:
a reduced mean total number of gadolinium-enhancing lesions as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon,
a reduced annualized relapse rate as compared to untreated patients and/or as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon,
a longer time to reach 3-month confirmed disability progression as compared to patients receiving another disease-modifying treatment selected from interferon, teriflunomide, glatiramer acetate and dimethyl fumarate, preferably interferon, teriflunomide and dimethyl fumarate, more preferably teriflunomide or interferon,
within up to 24 months, preferably 12-24 months of treatment.

25. The method according to claim 1, wherein by week 12 or by week 24 of treatment the levels of alanine aminotransferase (ALT), aspartate aminotransferase (AST), and lipase do not change by more than 10% as compared to the baseline level at the start of therapy.

26. The method according to claim 1, wherein the treatment is at least as effective in reducing the annual relapse rate as a CD20-depleting therapy.

27-39. (canceled)
